(19) United States
(12) Patent Application Publication
(10) Pub. No.: US 2026/0152797 A1
(43) Pub. Date: Jun. 4, 2026

(54) EPITHELIAL-MESENCHYMAL TRANSITION-BASED GENE EXPRESSION SIGNATURE FOR KIDNEY CANCER

(71) Applicant: THE REGENTS OF THE UNIVERSITY OF MICHIGAN, Ann Arbor, MI (US)

(72) Inventors: Aaron Udager, Ann Arbor, MI (US); Randy Vince, Ann Arbor, MI (US); Srinivas Nallandhighal, Ann Arbor, MI (US); Simpa Saiami, Ann Arbor, MI (US)

(21) Appl. No.: 18/706,540

(22) PCT Filed: Nov. 2, 2022
(86) PCT No.: PCT/US2022/048678

§ 371 Date: May 1, 2024

Related U.S. Application Data
(60) Provisional application No. 63/274,597, filed on Nov. 2, 2021.

(51) Int. Cl. C12Q 1/6886

(52) U.S. Cl. CPC C12Q 1/6886; C12Q 2600/118; C12Q 2600/158

(57) ABSTRACT

Provided herein are markers and methods to improve management of renal cancer. In some embodiments, the disclosure relates to a panel of biomarkers and use thereof to improve management of renal cell carcinoma, including clear cell renal cell carcinoma.

### STATEMENT REGARDING RELATED APPLICATIONS

This application claims priority to U.S. Provisional Patent Application No. 63/274,597, filed Nov. 2, 2021, the entire contents of which are incorporated herein by reference for all purposes.

### FIELD

The present disclosure relates to markers and methods to improve management of renal cancer. In some embodiments, the disclosure relates to a panel of biomarkers and use thereof to improve management of renal cell carcinoma, including clear cell renal cell carcinoma.

### BACKGROUND

Renal cell carcinoma (RCC) accounts for approximately 4.2% of all newly diagnosed cancer cases in the United States annually. Although surgery is curative in most patients, approximately 20% experience recurrence or distant metastasis.¹ Further, of all newly diagnosed cases, approximately 10% present with advanced disease displaying a tumor thrombus extending into the inferior vena cava (IVC).² The presence of IVC tumor thrombus significantly limits overall survival independently of other prognostic disease features such as tumor size, fat invasion, lymph node invasion, or distant metastasis.³ To improve on RCC survival outcomes, there is a need to understand the molecular underpinnings across the spectrum of RCC progression and identify critical molecular pathways that result in disease recurrence.

### SUMMARY

In some aspects, provided herein are methods. In some embodiments, the methods comprise determining expression of a panel of genes in a sample obtained from a subject. In some embodiments, provided here in is method comprising determining expression of a panel of genes in a sample obtained from a subject, wherein the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the panel of genes comprises at least 6 genes. In some embodiments, the panel of genes comprises at least 10 genes. In some embodiments, the panel of genes comprises at least 14 genes. In some embodiments, the panel of genes comprises at least 18 genes. In some embodiments, the panel of genes comprises each of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the panel of genes comprises less than 50 genes. For example, in some embodiments the panel comprises at least 4 genes and less than 50 genes, wherein the genes are selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the method further comprises determining expression of one or more genes involved in cell cycle proliferation. In some embodiments, the one or more genes involved in cell cycle proliferation are selected from FOXM1, ASPM, TK1, PRC1, CDC20, BUB1B, PBK, DTL, CDKN3, RRM2, ASF1B, CEP55, CDC2, DLGAP5, C18orf24, RAD51, KIF11, BIRC5, RAD54L, CENPM, KIAA0101, KIF20A, PTTG1, CDCA8, NUSAP1, PLK1, CDCA3, ORC6L, CENPF, TOP2A, and MCM10. In some embodiments, the method comprises determining the expression of less than 100 genes in total.

In some embodiments, the method comprises determining the expression of less than 60 genes in total.

In some embodiments, the method further comprises determining expression of one or more housekeeping genes in the sample, and normalizing the expression of each member of the panel of genes using the expression of the one or more housekeeping genes. For example, in some embodiments the one or more housekeeping genes are selected from ATP5E, ARF1, CLTC1, and PGK1.

Expression of the panel of genes may be determined by any suitable method. In some embodiments, expression of the panel of genes is determined by quantitative PCR (q-PCR).

In some embodiments, the method further comprises assigning an epithelial-mesenchymal transition (EMT) score to the subject based upon the expression of the panel of genes. In some embodiments, a EMT score is indicative of increased expression of the panel of genes compared to expression of the equivalent panel of genes for a low EMT score.

In some embodiments, the sample is a tumor sample. In some embodiments, the subject is a human. In some embodiments, the subject is suspected of having or at risk of having renal cancer. In some embodiments, the subject has received a first treatment regimen for renal cancer. For example, in some embodiments the first treatment regimen for renal cancer comprises a surgical procedure. In some embodiments, the renal cancer is clear cell renal cell carcinoma.

In some aspects, provided herein are methods of predicting disease outcome in a subject. In some embodiments, the method comprises determining expression of a panel of genes in a sample obtained from the subject. In some embodiments, the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the panel of genes comprises at least 6 genes. In some embodiments, the panel of genes comprises at least 10 genes. In some embodiments, the panel of genes comprises at least 14 genes. In some embodiments, the panel of genes comprises at least 18 genes. In some embodiments, the panel comprises each of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the panel of genes comprises less than 50 genes. For example, in some embodiments the panel comprises at least 4 genes and less than 50 genes, wherein the genes are selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

In some embodiments, the method further comprises determining expression of one or more housekeeping genes in the sample, and normalizing the expression of each member of the panel of genes using the expression of the one or more housekeeping genes. In some embodiments, the one or more housekeeping genes are selected from ATP5E, ARF1, CLTC1, and PGK1. In some embodiments, the method comprises determining the expression of less than 100 genes in total. In some embodiments, the method comprises determining the expression of less than 60 genes in total.

In some embodiments, expression of the panel of genes is determined by quantitative PCR (q-PCR). In some embodiments, the method comprises predicting poor disease outcome in the subject when expression of the panel of genes is elevated in the sample. In some embodiments, a poor disease outcome comprises reduced progression free survival (PFS) and/or disease specific survival (DSS) in the subject.

In some embodiments, the method further comprises assigning an epithelial-mesenchymal transition (EMT) score to the subject based upon the expression of the panel of genes. In some embodiments, a high EMT score is indicative of increased expression of the panel of genes compared to expression of the equivalent panel of genes for a low EMT score. In some embodiments, a high EMT score is predictive of reduced progression free survival (PFS) and/or disease specific survival (DSS) in the subject. In some embodiments, the EMT score is generated by log 2 transforming the mean expression for each gene in the panel. In some embodiments, an EMT score above a cutoff value of 1.22 is predictive of reduced progression free survival (PFS) and/or disease specific survival (DSS) in the subject compared to PFS and/or DSS in subjects having EMT scores below the cutoff value.

In some embodiments, the subject has received a first treatment regimen for renal cancer. In some embodiments, the first treatment regimen comprises a surgical procedure. In some embodiments, the renal cancer is clear cell renal cell carcinoma. In some embodiments, the method comprises treating the subject with an aggressive cancer treatment regimen when poor disease outcome is predicted. In some embodiments, the aggressive cancer treatment regimen comprises one or more therapies selected from radiation therapy, immunotherapy, chemotherapy, targeted therapy, and combinations thereof.

In some aspects, provided herein are methods of treating a subject. In some embodiments, provided herein is a method of treating a subject comprising determining expression of a panel of genes in a sample obtained from the subject and treating the subject with an appropriate treatment regimen based upon expression of the panel of genes. In some embodiments, the subject has received a first treatment regimen for renal cancer. In some embodiments, the first treatment regimen comprises a surgical procedure. In some embodiments, the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the method comprises assigning an epithelial-mesenchymal transition (EMT) score to the subject based upon the expression of the panel of genes. In some embodiments, a high EMT score is indicative of increased expression of the panel of genes compared to expression of the equivalent panel of genes for a low EMT score. In some embodiments, the method comprises treating the subject with an aggressive cancer treatment regimen when the EMT score is above a cutoff value. In some embodiments, the aggressive cancer treatment regimen comprises one or more therapies selected from radiation therapy, immunotherapy, chemotherapy, targeted therapy, and combinations thereof.

In some aspects, provided herein are kits. In some embodiments, provided herein is a kit comprising reagents for detecting one or more genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the kit additionally comprises reagents for detecting one or more genes involved in cell cycle proliferation. In some embodiments, the one or more genes involved in cell cycle proliferation are selected from FOXM1, ASPM, TK1, PRC1, CDC20, BUB1B, PBK, DTL, CDKN3, RRM2, ASF1B, CEP55, CDC2, DLGAP5, C18orf24, RAD51, KIF11, BIRC5, RAD54L, CENPM, KIAA0101, KIF20A, PTTG1, CDCA8, NUSAP1, PLK1, CDCA3, ORC6L, CENPF, TOP2A, and MCM10. In some embodiments, the kit detects less than 100 genes in total. In some embodiments, the kit detects less than 60 genes in total.

### DESCRIPTION OF THE DRAWINGS

FIGS. 1A-1H show integrative molecular analyses of ccRCC in the discovery cohorts. FIG. 1A is a schematic showing molecular dissection of primary kidney cancers and synchronous inferior vena cava (IVC) tumor thrombi in the discovery cohort. Patients with ccRCC and synchronous IVC tumor thrombus (n=5) who had undergone radical nephrectomy with IVC tumor thrombectomy were retrospectively identified. Formalin-fixed paraffin-embedded (FFPE) specimens were retrieved; Hematoxylin and eosin (H&E) slides were reviewed, outlining three regions each from the primary tumors and IVC tumor thrombi for targeted RNA next generation sequencing (NGS, # of samples=30). FIG. 1B is a boxplot displaying derived cell cycle proliferation (mxCCP) scores. Tumor thrombi demonstrated higher CCP scores compared with matched primary ccRCC (unpaired two-sided t-test, p<0.01). FIG. 1C is a graph showing differential gene expression analyses. Paired differential expression analysis revealed over-expression of WT1 and proliferation genes in tumor thrombi compared to primary ccRCC tumors. Genes with Log-likelihood ratio test FDR<5% were deemed to be significantly differentially expressed. FIG. 1D is a boxplot displaying WT1 log 2 expression. Tumor thrombi demonstrated higher WT1 expression compared with matched primary ccRCC (unpaired two-sided t-test, p<0.01). FIG. 1E is a schematic showing molecular profiling of primary ccRCC with and without recurrence following nephrectomy. Patients who underwent nephrectomy for localized ccRCC and with follow up data available were retrospectively identified (n=36). Patients who developed recurrence were matched to patients without recurrence during follow up in ratio 1:1 based on tumor stage, grade, and duration of follow up. FFPE specimens were retrieved, H&E slides were reviewed and a region of tumor from each kidney was outlined for molecular analyses targeted RNA NGS. FIG. 1F is a boxplot displaying derived mxCCP scores. There was no significant difference between the CCP scores of patients with and without recurrent disease (unpaired two-sided t-test, p=0.15). FIG. 1G is a graph showing differential expression analysis, which revealed significant over-expression of WT1 and CCP genes in patients with disease recurrence (FDR<5%). FIG. 1H is a boxplot displaying WT1 log 2 expression. Patients who developed disease recurrence demonstrated higher WT1 expression compared with those without recurrence (unpaired two-sided t-test, p=0.05; The p<0.05 when the four patients without recurrence but with outlier WT1 expression were excluded).

FIGS. 2A-2D show interrogation of WT1 expression in the validation (TCGA) cohort. FIG. 2A and FIG. 2B show WT1 expression and survival analyses. WT1 expression values were stratified based on the logCPM optimal cut-point of WT1 expression into low (logCPM≤0.04) and high (logCPM>0.04). Kaplan-Meier (KM) survival analysis were performed using R package survival. WT1 over-expression was associated with worse progression free survival (PFS) and poorer disease specific survival (DSS), as shown. FIG. 2C and FIG. 2D are graphs showing molecular alterations/pathway enrichment associated with WT1 over-expression. Differential expression analysis comparing high versus low WT1 tumors revealed 382 differentially expressed genes at an absolute log 2 fold-change (LFC) cut-off of 1 (2 in linear space) and FDR<5% (FIG. 2C). Pathway analysis using GSEA MSigDB Hallmark pathways was performed by R package fgsea using ranked log 2 fold-change values (FIG. 2D). The most significant pathways at a Benjamini-Hochberg (BH)-adjusted FDR<5% are displayed in descending order of normalized enrichment score (NES). Notably, tumors with high WT1 expression were associated with enrichment in EMT, E2F targets, G2M checkpoint and other key angiogenic and inflammatory pathways.

FIGS. 3A-3D show development and evaluation of a novel epithelial-mesenchymal transition (EMT) score in the validation (TCGA) cohort. FIG. 3A is a graph showing differential expression analysis using 195 GSEA Hallmark EMT pathway genes, which revealed 22 over-expressed genes at an absolute log 2fold-change (LFC) cut-off of 1 (2 on a linear scale) and FDR<5% among tumors that progressed/recur compared to tumors that did not recur. FIG. 3B shows unsupervised hierarchical clustering of the 22 over-expressed EMT genes. Each column represents a patient's sample with progression free survival (PFS) status, tumor grade and stage as shown. The 22 over-expressed genes were used to generate a novel EMT score. FIG. 3C and FIG. 3D show EMT score and survival analyses. Kaplan-Meier survival analysis was performed using R package survival. EMT scores were stratified based on optimal cut-point into low (score≤1.22) and high (score>1.22) scores. Higher EMT scores were associated with worse PFS and poorer disease specific survival (DSS), as shown.

FIGS. 4A-4D show synergistic prognostic implication of CCP and EMT pathways enrichment in the validation (TCGA) cohort. FIG. 4A and FIG. 4B show novel EMT score and derived CCP score and survival analyses. Youden index cut-point values were determined and used to stratify tumors into low and high CCP (cut-off=0.7) as well as low and high EMT (cut-off=1.22). Next, all tumors were stratified into four risk groups: CCP^{low}/EMT^{low}; CCP^{low}/EMT^{high}; CCP^{high}/EMT^{low}; and CCP^{high}/EMT^{high}. Kaplan-Meier survival analysis was then performed demonstrating the worst progression free survival (PFS) and poorest disease specific survival (DSS) in CCP^{high}/EMT^{high} risk group. FIG. 4C and FIG. 4D show multivariable Cox proportional hazard analyses. Adjusting for clinicopathologic variables, high CCP and EMT scores were significantly associated with PFS and DSS, with the CCP^{high}/EMT^{high} risk group having the worst outcome. Addition of CCP and EMT scores to clinicopathologic variables in multivariable Cox proportional hazard models improved the concordance indices from 0.73 to 0.78 for predicting PFS (FIG. 11A) and 0.77 to 0.84 for predicting DSS (FIG. 11B).

FIGS. 5A-5C show RNAseq analyses in the discovery cohorts. FIG. 5A shows unsupervised hierarchical clustering of median-centered expression of 130 genes (columns) and 24 samples (rows), which revealed patient specific clustering pattern in discovery cohort-A. Sample clustering was performed using ward. D2 method and genes were clustered using Pearson correlation. FIG. 5B shows unsupervised hierarchical clustering of median-centered expression of 130 genes (columns) and 36 samples (rows), which revealed a combination of progression and grade specific clustering pattern in discovery cohort-B. Sample clustering was performed as described in panel A. FIG. 5C shows Kaplan-Meier survival analysis of normalized WT1 expression performed in discovery cohort-B using progression free survival (PFS) status. A cut-point value of −0.24 was used to stratify tumors into low and high groups. High WT1 was significantly associated with worse PFS (p=0.049) as shown.

FIGS. 6A-6D show derivation and evaluation of cell cycle proliferation (CCP) score in the validation (TCGA) cohort. FIG. 6A shows unsupervised hierarchical clustering of 31 CCP genes, which reveal clusters with distinct proliferation pattern. Gene clustering was performed using Pearson correlation and sample clustering was performed using ward. D2 method. Each column represents a patient's sample with progression free survival (PFS) status, tumor grade and stage as shown. FIG. 6B shows boxplots displaying higher derived mxCCP scores pathologic stage T3-4 compared with T1-2 tumors (left panel) as well as higher derived mxCCP scores in grade 3-4 compared with grade 1-2 tumors (right panel). Two-sided t-test p-values were calculated for each comparison to determine statistical significance. FIG. 6C and FIG. 6D show Kaplan-Meier survival analysis performed using R package survival. Derived mxCCP scores were stratified based on optimal cut-point into low (score≤0.7 and high (score>0.7) scores. Higher derived mxCCP scores were associated with worse PFS and poorer disease specific survival (DSS), as shown.

FIGS. 7A-7F show Multivariable Cox proportional hazard model analyses adjusting for relevant clinicopathologic variables in the validation (TCGA) cohort. FIG. 7A and FIG. 7B show High mxCCP scores were independently associated with PFS (p<0.001) and DSS (p<0.001).

FIG. 7C and FIG. 7D show WT1 expression was not significantly associated with PFS or DSS (p=0.05 and p>0.05). FIG. 7E and FIG. 7F show high EMT scores were significantly associated with PFS and DSS (both p<0.001).

FIGS. 8A-8D show distribution of WT1 expression and EMT score in the validation (TCGA) cohort. FIG. 8A and FIG. 8B are boxplots displaying higher WT1 expression in pathologic stage T3-4 compared with T1-2 tumors as well as higher WT1 expression in Fuhrman grade 3-4 compared with grade 1-2 tumors (both p<0.001). FIG. 8C and FIG. 8D are boxplots displaying higher derived EMT scores in pathologic stage T3-4 compared with T1-2 tumors as well as higher derived EMT scores in Fuhrman grade 3-4 compared with grade 1-2 tumors (both p<0.001). Two-sided t-test p-values were calculated for each comparison to determine statistical significance.

FIG. 9 shows a correlation heatmap of genes from the GSEA MSigDB Epithelial-mesenchymal transition (EMT) hallmark pathway. Correlation heatmap of 22 differentially overexpressed EMT genes in WT1^{high} versus WT1^{low} tumors which were used to derive the novel EMT score in the validation (TCGA) ccRCC cohort. Numbers denote Pearson correlation coefficient values.

FIGS. 10A-10B show association of WT1 expression with E-Cadherin protein expression and derived EMT score in the validation (TCGA) cohort. FIG. 10A is a boxplot comparing E-cadherin protein expression (CDH1 RPPA z-score) across low and high WT1 expression. As WT1 expression increases, a significant decreasing trend in E-cadherin protein expression, a marker of EMT, was observed (p<0.001). FIG. 10B is a boxplot showing a significant increasing trend in derived EMT score was observed across low and high WT1 expression (p<0.001). Comparisons were made using a two-sided t-test.

FIG. 11A-11B show Multivariable Cox proportional hazard model analyses of relevant clinicopathologic variables in the validation (TCGA) cohort. FIG. 11A shows that age, sex, and tumor stage were independently associated with PFS (p<0.05). FIG. 11B shows that age and tumor stage were independently associated with DSS (p<0.05).

FIG. 12 is a graph showing Kaplan-Meier survival analysis demonstrating a significant decrease in metastasis-free survival in patients with a high EMT score.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. In some embodiments, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification, and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems.

The terms “sample” and “biological sample” are used interchangeably to refer to any biological sample obtained from an individual including body fluids, body tissue (e.g., tumor tissue), cells, or other sources. Body fluids are, for example, blood and blood products (e.g. whole blood, capillary blood, venous blood, plasma, serum, etc.), urine, saliva, semen, synovial fluid, and spinal fluid. Samples also include tissue, such as tumor tissue. Methods for obtaining tissue biopsies and body fluids from mammals are well known in the art. In some embodiments, the sample is a “tumor sample”. A “tumor sample” herein is a sample derived from, or comprising tumor cells from a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

As used herein, the terms “treat,” “treatment,” and “treating” refer to reducing the amount or severity of a particular condition, disease state (e.g., cancer), or symptoms thereof, in a subject presently experiencing or afflicted with the condition or disease state. The terms do not necessarily indicate complete treatment (e.g., total elimination of the condition, disease, or symptoms thereof). In some embodiments, “treating” cancer refers to reducing the size of a tumor, reducing the number of tumors, and/or completely eliminating the tumor from a subject. “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

### DETAILED DESCRIPTION

In some aspects, provided herein are methods. In some embodiments, provided herein are methods comprising determining expression of a panel of genes in a sample obtained from a subject. In some embodiments, the subject is a human. In some embodiments, the subject is suspected of having or at risk of cancer. In some embodiments, the subject is suspected of having or at risk of having renal cancer. In some embodiments, the method comprises determining expression of a panel of genes in a sample obtained from a subject having or suspected of having renal cancer. In some embodiments, the subject has received a first treatment regimen for renal cancer. In some embodiments, the treatment regimen comprises surgery. In some embodiments, provided herein is a method comprising determining expression of a panel of genes in a sample obtained from a subject, wherein the subject has received a first treatment regimen for renal cancer.

Gene expression can be determined either at the RNA level (i.e., mRNA or noncoding RNA (ncRNA)) (e.g., miRNA, tRNA, rRNA, snoRNA, siRNA and piRNA) or at the protein level. In some embodiments, measuring gene expression at the mRNA level includes measuring levels of cDNA corresponding to mRNA. In some embodiments, determining expression of a gene comprises determining an RNA level for the gene. In some embodiments, determining expression a gene comprises determining a level of a protein encoded by the gene. Various suitable methods for determining expression of a gene may be employed. Suitable techniques for determining gene expression include, but are not limited to, sequencing techniques (including DNA sequencing and RNA sequencing techniques), amplification based techniques such as polymerase chain reaction (PCR) based techniques (e.g. PCR, reverse transcription PCR (RT-PCR), qualitative PCR (qPCR), digital PCR, droplet digital PCR), hybridization techniques (e.g. in situ hybridization, fluorescence in situ hybridization, microarray, Southern blot, Northern blot), serial analysis of gene expression (SAGE), Digital Gene Expression (DGE), and immunoassays (e.g. immunoprecipitation, Western blot, ELISA, immunohistochemistry, immunocytochemistry, flow cytometry, immune-PCR, etc.).

In some embodiments, the panel of genes comprises one or more selected from Dickkopf WNT signaling pathway inhibitor 1 (DKK1), collagen type VII alpha 1 chain (COL7A1), collagen type VIII alpha 2 chain (COL8A2), cartilage oligomeric matrix protein (COMP), secreted frizzled-related protein 4 (SRFP4), collagen type XI alpha 1 chain (COL11A1), leucine rich repeat containing 15 (LRRC15), lumican (LUM), paired related homeobox 1 (PRRX1), collagen type 1 alpha 1 chain (COL1A1), collagen type VI alpha 3 chain (COL6A3), growth arrest specific 1 (GAS1), procollagen C-endopeptidase enhancer (PCOLCE), lysyl oxidase like 1 (LOXL1), matrix remodeling associated 5 (MXRA5), fibrillin 2 (FBN2), tissue factor pathway inhibitor 2 (TFPI2), interleukin 6 (IL6), pentraxin 3 (PTX3), collagen triple helix repeat containing 1 (CTHRC1), serpin family E member 1 (SERPINE1), and lysyl oxidase like 2 (LOXL2). It is contemplated that these genes are involved in epithelial-mesenchymal transition (EMT) and are also referred to herein as an “EMT panel”. An epithelial-mesenchymal transition (EMT) is a biologic process that allows a polarized epithelial cell, which normally interacts with basement membrane via its basal surface, to undergo multiple biochemical changes that enable it to assume a mesenchymal cell phenotype, which includes enhanced migratory capacity, invasiveness, elevated resistance to apoptosis, and greatly increased production of ECM components. Accordingly, EMTs are recognized as mechanisms for initiating the invasive and metastatic behavior of cancers. In some embodiments, the panel of genes comprises at least 2 genes, at least 3 genes, at least 4 genes, at least 5 genes, at least 6 genes, at least 7 genes, at least 8 genes, at least 9 genes, at least 10 genes, at least 11 genes, at least 12 genes, at least 13 genes, at least 14 genes, at least 15 genes, at least 16 genes, at least 17 genes, at least 18 genes, at least 19 genes, at least 20 genes, or least 21 genes. In some embodiments, the panel comprises each of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

In some embodiments, the method further comprises determining expression of one or more housekeeping genes. As used herein, the term “housekeeping genes” or “normalizing genes” are used interchangeably to refer to the genes whose expression is used to calibrate or normalize the measured expression of the gene of interest (e.g., a gene in the panel of genes). The expression of housekeeping genes should be independent of cancer outcome/prognosis, and the expression of the housekeeping genes should be highly similar among all of the samples. This normalization helps to ensure accurate comparison of expression of the gene panels between different samples.

Any suitable housekeeping gene(s) known in the art can be used. One or more housekeeping genes can be used. In some embodiments, the housekeeping genes are selected from ATP synthase F1 subunit epsilon (ATP5E), ADP-ribosylation factor 1 (ARF1), clathrin heavy chain 1 (CLTC1), and phosphoglycerate kinase 1 (PGK1). In some embodiments, one housekeeping gene is used. In some embodiments, 2 housekeeping genes are used. In some embodiments, 3 housekeeping genes are used. In some embodiments, each of ATP5E, ARF1, CLTC1, and PGK1 are used.

In some embodiments, a score is generated based upon the expression (e.g. the normalized expression) of the panel of genes. In some embodiments, a score is generated based upon the normalized expression of each member in the panel of genes. A score may be generated, at least in part, by combining the normalized expression for each member of the panel of genes. In some embodiments, the total normalized expression for the panel may be calculated (e.g. by adding the normalized expression of each gene to receive a total score). In some embodiments, the average normalized expression for the panel may be calculated. For example, the normalized expression of each member of the panel of genes may be combined, and that total may be divided by the number of genes to determine the average normalized expression across all genes in the panel. In some embodiments, each member in the panel receives equal weight. In some embodiments, one or more members of the panel is more significant than others (e.g. receives more weight than others). For example, one member of the panel may be at least 10%, 15%, 20%, 25%, 30%, 35%, 40%, 45%, 50%, 55%, 60%, 65%, 70%, 75%, 80%, 85%, 90%, or more than 90% of the total weight given to all of the genes in the panel.

In some embodiments, the score is referred to as an EMT score. In some embodiments, the method further comprises assigning an epithelial-mesenchymal transition (EMT) score to the subject based upon the expression of the panel of genes. Generally speaking, higher EMT scores are indicative of increased expression (e.g. increased average expression) of the panel of genes in the subject compared to expression of the same panel of genes in a second subject. Accordingly, a higher EMT score is indicative of increased average expression of a panel of genes comprising one or more of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2 in the subject.

In some embodiments, RNA levels for the genes are measured, and subsequently used for assigning a score (e.g. an EMT score) to the subject. In some embodiments, RNA levels for the genes are measured by a reverse transcription reaction to generate cDNA, followed by a quantitative PCR (qPCR) assay. Typically, a cycle threshold (Ct) value is determined for each test gene and each normalizing (e.g. housekeeping) gene. The Ct value indicates the number of cycles at which the fluorescence from a qPCR reaction above background is detectable. The Ct value for each gene may be normalized by subtracting the Ct value for a housekeeping gene or the average Ct value for multiple housekeeping genes. The normalized values may be converted to an expression value for the gene, which may be an estimate of the copy number for that gene. In some embodiments, the mean expression value for each gene may be averaged. The average expression value may be transformed, such as by a base 2 algorithm (e.g. log 2 transformation), to generate the EMT score for the subject.

Expression of the panel of genes may be measured from any suitable sample type obtained from the subject. In some embodiments, the sample is a bodily fluid such as blood (e.g. whole blood, capillary blood, venous blood), plasma, serum, urine, saliva, semen, synovial fluid, or spinal fluid. In some embodiments, the sample is a tissue sample. In some embodiments, the sample is a tumor sample. The tumor sample may be any sample derived from or comprising cells from a tumor in the subject. For example, the tumor sample may be a tumor biopsy, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In some embodiments, the expression levels of genes identified herein are measured in tumor tissue. For example, the tumor tissue may be obtained upon surgical resection of the tumor, or by tumor biopsy. The expression level of the identified genes may also be measured in tumor cells recovered from sites distant from the tumor, including circulating tumor cells or body fluid (e.g., urine, blood, blood fraction, etc.).

In some embodiments, the subject is afflicted with or at risk of developing cancer. The terms “cancer” and “carcinoma” refer to or describe the physiological condition in mammals that is typically characterized by unregulated cell growth. The pathology of cancer includes, for example, abnormal or uncontrollable cell growth, metastasis, interference with the normal functioning of neighboring cells, release of cytokines or other secretory products at abnormal levels, suppression, or aggravation of inflammatory or immunological response, neoplasia, premalignancy, malignancy, invasion of surrounding or distant tissues or organs, such as lymph nodes, blood vessels, etc. In some embodiments, the cancer is renal cancer. As used herein, the terms “renal cancer” or “renal cell carcinoma” refer to cancer that has arisen from the kidney. Renal cancer encompasses several histologic subtypes, including clear cell renal cell carcinoma (ccRCC), papillary renal cell carcinoma (pRCC), chromophobic renal cell carcinoma, collecting duct renal cell carcinoma (cdRCC), medullary carcinoma, transitional cell carcinoma (TCC), Wilms tumor (WT), renal sarcoma (RS), or unclassified renal cell carcinoma (RCC). In some embodiments, the renal cancer is clear cell renal cell carcinoma (ccRCC). In some embodiments, the subject has received a first treatment regimen for renal cancer (e.g. ccRCC). For example, in some embodiments the subject has received surgery as a first treatment regimen for renal cancer.

In some embodiments, the expression of the panel of genes is predictive of cancer outcome in the subject. For example, the expression of the panel of genes, and/or the score assigned to the subject, may be indicative of cancer prognosis (e.g. ccRCC prognosis). In some embodiments, an elevated score is indicative of poor prognosis for the subject. In some embodiments, an elevated EMT score is indicative of poor prognosis for the subject. As used herein, the term “poor prognosis” is used to indicate an undesirable cancer outcome, including increased likelihood of cancer recurrence, increased likelihood of metastasis, reduced progression free survival (PFS), reduced disease-specific survival (DFS), or a combination thereof. In some embodiments, an EMT score above a threshold value is indicative of poor prognosis. In some embodiments, an EMT score is considered “high” when the score is above 1.22. In some embodiments, an EMT score is considered “low” when the score is equal to or below 1.22. A high EMT score (e.g. a score above 1.22) may be indicative of poor prognosis in the subject, including reduced PFS and/or reduced DFS in the subject. In some embodiments, expression of the panel of genes is predictive of cancer outcome following a first treatment regimen in the subject. For example, in some embodiments, expression of the panel of genes is predictive of cancer outcome following a surgical procedure in the subject. In some embodiments, “poor prognosis” may be indicative of increased likelihood of cancer recurrence, reduced progression free survival, reduced disease-specific survival, or a combination thereof following a surgical procedure (e.g. a nephrectomy) in the subject.

In some embodiments, the methods described herein further comprise measuring expression of one or more additional genes. For example, expression of one or more additional genes may be measured in a sample obtained from the subject. For example, expression of one or more genes involved in cell cycle proliferation may be measured. In some embodiments, the one or more additional genes involved in cell cycle proliferation are selected from forkhead box protein M1 (FOXM1, assembly factor for spindle microtubules (ASPM), thymidine kinase 1 (TK1), protein regulator of cytokinesis 1 (PRC1), cell division cycle 20 (CDC20), BUB1 mitotic checkpoint serine/threonine kinase B (BUB1B), PDZ binding kinase (PBK), denticleless E3 ubiquitin protein ligase homolog (DTL), cyclin dependent kinase inhibitor 3 (CDKN3), ribonucleotide reductase regulatory subunit M2 (RRM2), anti-silencing function 1B histone chaperone (ASF1B), centrosomal protein 55 (CEP55), cyclin dependent kinase 1 (CDC2), DLG associated protein 5 (DLGAP5), spindle and kinetochore associated complex subunit 1 (C18orf24), RAD51 recombinase (RAD51), kinesin family member 11 (KIF11), baculoviral IAP repeat containing 5 (BIRC5), RAD54 like (RAD54L), centromere protein M (CENPM), PCNA clamp associated factor (KIAA0101), kinesin family member 20A (KIF20A), PTTG1 regulator of sister chromatic separation, securin (PTTG1), cell division cycle associated 8 (CDCA8), nucleolar and spindle associated protein 1 (NUSAP1), polo like kinase 1 (PLK1), cell division cycle associated 3 (CDCA3), origin recognition complex subunit 6 (ORC6L), centromere protein F (CENPF), DNA topoisomerase II alpha (TOP2A), and minichromosome maintenance 10 replication initiation factor (MCM10). In some embodiments, expression of one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, or all 31 of the cell cycle proliferation genes are measured. In some embodiments, one or more additional markers may be measured in the subject to assist in prognosing cancer outcome. In some embodiments, the above-described genes involved in cell cycle proliferation may be used to generate a cell cycle proliferation score (CCP) score, as described in Lancet Oncol. 2011 March; 12(3): 245-255, the entire contents of which are incorporated herein by reference for all purposes. In some embodiments, a combination of the EMT Score and the CCP score used for cancer prognosis in the subject.

In some embodiments, the methods further comprise selecting an appropriate treatment regimen for the subject. In some embodiments, the method further comprises selecting an appropriate treatment regimen for the subject following an initial treatment (e.g. a first treatment regimen) for the cancer. In some embodiments, the first treatment regimen for the cancer comprises a surgical procedure. Suitable surgical procedures include, for example, laparoscopic procedures, biopsy, or tumor ablation, such as cryotherapy, radio frequency ablation, and high intensity ultrasound. Additional suitable surgical procedures include nephrectomy, including a partial nephrectomy (e.g. a removal of the cancer within the kidney), a simple nephrectomy (e.g. removal of the kidney itself) or a radical nephrectomy (e.g. a procedure to remove the kidney, the adrenal gland, surrounding tissue, and sometimes nearby lymph nodes).

In some embodiments, the method comprises selecting an appropriate treatment regimen for the subject following the first treatment regimen that the subject has already received. The treatment regimen that is selected based upon expression of the panel of genes may be referred to herein as a “second treatment regimen”, a “second treatment”, a “follow up treatment”, or a “follow up treatment regimen”. In some embodiments the method comprises selecting an appropriate treatment regimen following a surgical procedure to treat renal cancer in the subject, such as a partial nephrectomy, a simple nephrectomy, or a radical nephrectomy. For example, a treatment regimen may be selected based upon expression of one or more genes in the panel and/or the score assigned to the subject. For example, a subject having elevated expression of the panel of genes may be identified as a subject that would benefit from one or more additional therapies following surgery, such as one or more of radiation therapy, immunotherapy, chemotherapy, targeted therapy, or combinations thereof.

Suitable treatment regimens include, for example, radiation therapy, immunotherapy, chemotherapy, targeted therapy, or combinations thereof. In some embodiments, a combination of treatment regimens may be employed. In some embodiments, the treatment regimen may comprise immunotherapy. Suitable immunotherapy includes, for example, cytokine immunotherapy, such as with interleukin-2 (IL-2). Other suitable immunotherapies include, for example, interferon therapy, or immune-checkpoint inhibitor therapies, such as PD-1 inhibitors (nivolumab, pembrolizumab, avelumab, etc.) or CTLA-4 inhibitors (e.g. ipilimumab.). In some embodiments, the treatment regimen comprises targeted therapy, such as treatment with antiangiogenic agents including kinase inhibitors or monoclonal antibodies. Suitable kinase inhibitors include mTOR inhibitors (e.g. everolimus, temsirolimus), and VEGF inhibitors (e.g. sunitinim, pazopanib, cabozantinib, axitinib, sorafenib, levantinib).

In some embodiments, the treatment regimen comprises therapy with an agent selected from everolimus, aldesleukin, bevacizumab, avelumab, axitinib, belzutifan, cabozantinib-S-Malate, tivozanib hydrochloride), IL-2 (Aldesleukin), ipilimumab, pembrolizumab, lenvatinib mesylate, sorafenib tosylate, nivolumab, pazopanib hydrochloride, sunitinib malate, temsirolimus, tivozanib hydrochloride, pazopanib hydrochloride, or belzutifan.

In some embodiments, an aggressive treatment regimen is selected for a subject having elevated expression of one or more genes in the panel. In some embodiments, an aggressive treatment regimen is selected for a subject having elevated expression of at least two genes in the panel. For example, an aggressive treatment regimen may be selected for a subject having elevated expression of two or more of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, an aggressive treatment regimen is selected for a subject determined as having a high EMT score and/or a high CPP score. As used herein, the term “aggressive treatment regimen” refers to a treatment at or above the standard level of treatment for the patient (e.g. at or above the standard level of treatment administered to the patient by a physician with knowledge of the patient's disease state). For example, in some embodiments a standard treatment regimen following surgery (e.g. following a nephrectomy) may comprise surveillance. For example, a patient not having an elevated score (e.g. not having a high EMT score and/or not having a high CPP score) may be a candidate for surveillance (e.g. monitoring of the cancer), whereas a patient having a high EMT score and/or a high CPP score may not be a good candidate for surveillance and may instead require an aggressive treatment regimen.

In some embodiments, an aggressive treatment regimen comprises a plurality of therapies. For example, an aggressive treatment regimen may comprise providing at least two treatment types to the subject. For example, an aggressive treatment regimen may comprise any combination of radiation therapy, immunotherapy, chemotherapy, and targeted therapy. In some embodiments, radiation therapy may be performed in addition to, for example, immunotherapy, chemotherapy, and/or targeted therapy. In some embodiments, an aggressive treatment regimen may comprise a high dose and/or frequent administration of the treatment to the subject compared to a less aggressive treatment regimen. Selection of the appropriate dosage and administration schedule may be performed by a physician, including a physician with knowledge of the subject's EMT and/or CCP score.

In some embodiments, provided herein is a method of treating a subject. In some embodiments, provided herein is a method of treating a subject, wherein the subject has received a first treatment regimen for cancer. In some embodiments, the cancer is renal cancer. In some embodiments, the first treatment regimen for renal cancer comprises a nephrectomy (e.g. a partial nephrectomy, a simple nephrectomy, or a radical nephrectomy). The methods comprise determining expression of a panel of genes as described herein. In some embodiments, the methods of treating a subject further comprise assigning a score to the patient based upon expression of the panel of genes. Suitable methods for assigning a score to the patient are described above. In some embodiments, the score is an EMT score. The methods further comprise treating the patient with an appropriate treatment regimen based upon expression of the panel of genes and/or based upon the score assigned to the subject. In some embodiments, the methods further comprise treating the patient with an aggressive treatment regimen, as described above, when the EMT score is above a cutoff value.

In some embodiments, provided herein are kits. In some embodiments, provided herein is a kit for determining expression of one or more genes in a panel as described herein. In some embodiments, provided herein is a kit comprising reagents for detecting one or more or each of the genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2. In some embodiments, the kit comprises reagents for detecting one or more of each of the genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2, along with one or more housekeeping genes. In some embodiments, the housekeeping genes are one or more of ATP5E, ARF1, CLTC1, and PGK1. In some embodiments, the kit additionally comprises reagents for detecting one or more genes involved in cell cycle proliferation. For example, in some embodiments the kit additionally comprises reagents for detecting one or more genes involved in cell cycle proliferation selected from FOXM1, ASPM, TK1, PRC1, CDC20, BUB1B, PBK, DTL, CDKN3, RRM2, ASF1B, CEP55, CDC2, DLGAP5, C18orf24, RAD51, KIF11, BIRC5, RAD54L, CENPM, KIAA0101, KIF20A, PTTG1, CDCA8, NUSAP1, PLK1, CDCA3, ORC6L, CENPF, TOP2A, and MCM10. In some embodiments, the kit comprises reagents for detecting less than 100 genes in total. For example, in some embodiments the kit comprises reagents for detecting one or more or each of the genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2, wherein the kit detects less than 100 genes in total (e.g. less than 100 genes, less than 75 genes, less than 50 genes, less than 40 genes, less than 30 genes, less than 25 genes, etc.).

The kit may comprise reagents suitable for determining expression of the one or more genes by any suitable technique, including mRNA-based and protein-based detection. For example, suitable techniques for determining gene expression include sequencing techniques (including DNA sequencing and RNA sequencing techniques), amplification based techniques such as polymerase chain reaction (PCR) based techniques (e.g. PCR, reverse transcription PCR (RT-PCR), qualitative PCR (qPCR), digital PCR, droplet digital PCR), hybridization techniques (e.g. in situ hybridization, fluorescence in situ hybridization, microarray, Southern blot, Northern blot), serial analysis of gene expression (SAGE), Digital Gene Expression (DGE), and immunoassays (e.g. immunoprecipitation, Western blot, ELISA, immunohistochemistry, immunocytochemistry, flow cytometry, immune-PCR, etc.). In some embodiments, the kit comprises oligonucleotides for detecting one or more genes in a panel along with one or more housekeeping genes. In some embodiments, the kit may comprise oligonucleotides, buffers, salts, preservatives, inhibitors (e.g. RNase inhibitors) dNTPs, enzymes, co-factors, primers, probes, and the like. In some embodiments, the kit comprises antibodies for detection of protein, along with suitable additional reagents including buffers, salts, preservatives, inhibitors (e.g. protease inhibitors), enzymes, stabilizers, and the like. In some embodiments, the kit additionally comprises instructions for use. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. The various components of the kit optionally are provided in suitable containers as necessary. The kit can further include containers for holding or storing a sample. Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

### EXAMPLES

### Example 1

Comprehensive genomic characterization of kidney cancer has uncovered several genetic alterations associated with clear cell RCC (ccRCC), such as VHL, PBRM1, SETD2, and BAP1 mutations. Additionally, large scale molecular profiling efforts, such as The Cancer Genome Atlas (TCGA) has led to the identification of key biologic processes in RCC. In clear cell RCC (ccRCC), the most common histologic subtype of kidney cancer, remodeling of cellular metabolism has been shown to be associated with higher-stage, high-grade, and poorer survival disease.⁴ However, molecular profiling is not routinely utilized for RCC diagnosis, prognostic risk stratification, or selection of targeted therapy. Prior work in ccRCC has focused on cell cycle proliferation (CCP) which was shown to provide a modest improvement in risk stratification of patients with ccRCC.⁵ The CCP classifier is a RNA expression assay that primarily measures the activity of genes involved in cellular proliferation and provides prognostic information beyond tumor grade and stage.⁶ However, additional molecular pathways may further improve stratifications of patients with ccRCC into appropriate risk categories.

Here, molecular dissection of locally advanced RCC with synchronous IVC thrombus as well as patients with localized disease who underwent radical nephrectomy with long-term follow up was performed to discern and validate molecular pathways associated with ccRCC progression and identify potential novel therapeutic targets.

### Materials and Methods

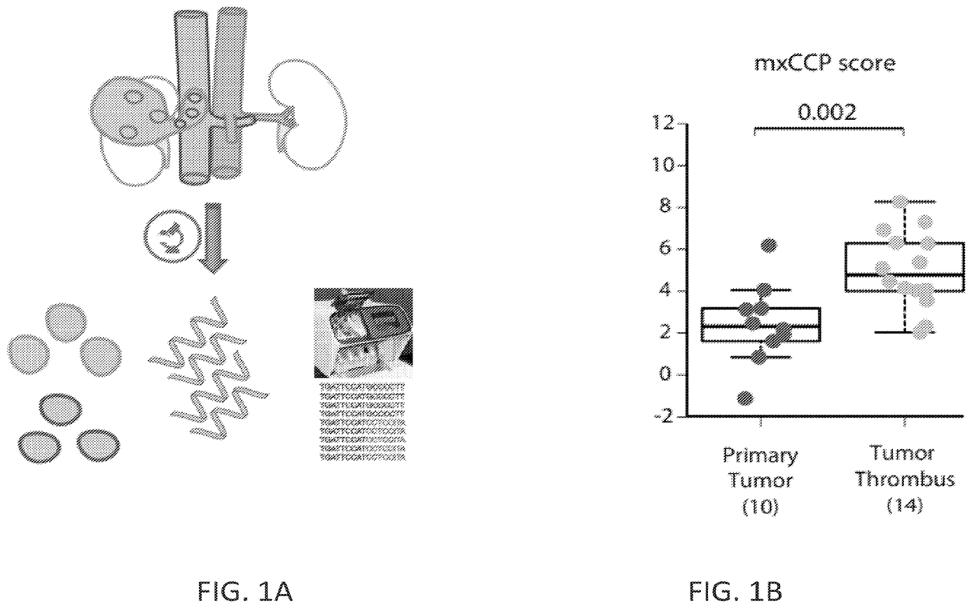

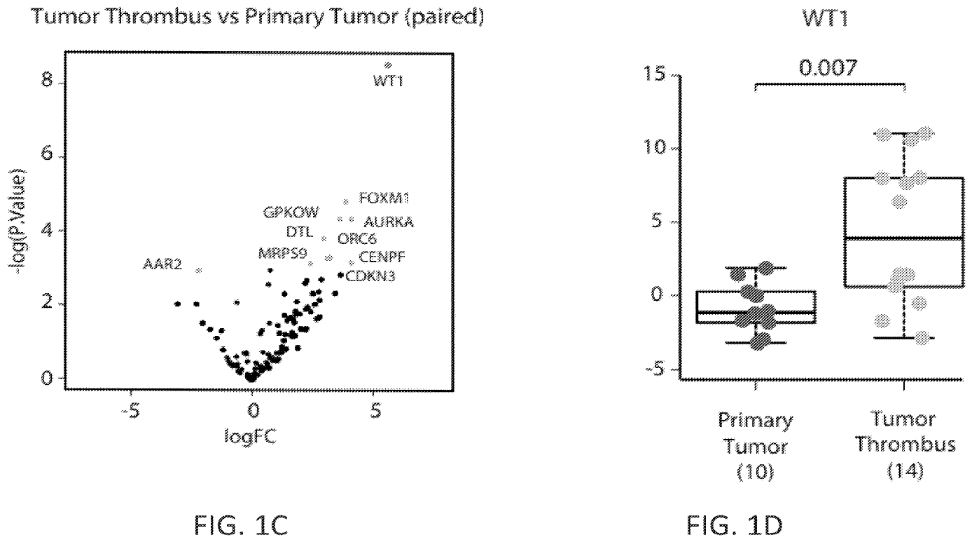

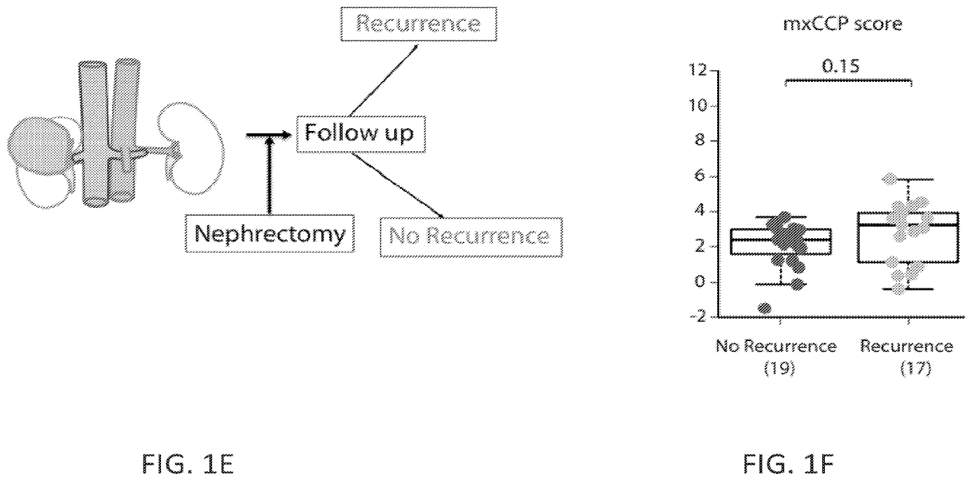

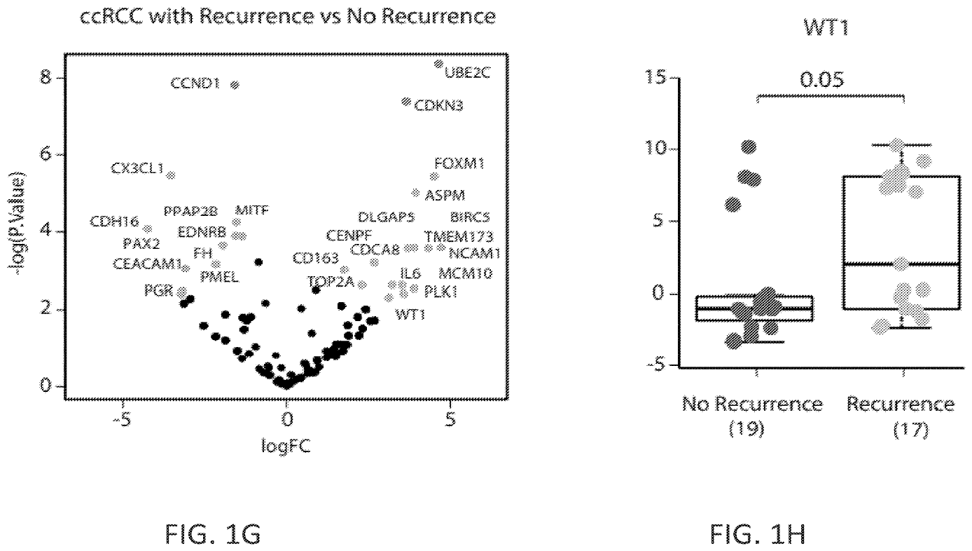

Discovery cohorts: Following institutional Internal Review Board approval two patient cohorts representing the spectrum of localized and locally advanced ccRCC were retrospectively identified (FIG. 1). Discovery cohort-A (n=5) were patients who underwent radical nephrectomy (RNx) with IVC tumor thrombectomy for ccRCC and with sufficient tissue available for analysis. Formalin-fixed paraffin-embedded (FFPE) specimens were retrieved, and a genitourinary pathologist reviewed the corresponding hematoxylin and eosin (H&E) slides, selecting three representative regions each from the primary and IVC tumor thrombus for analyses. Discovery cohort-B (n=36) underwent RNx for clinically localized ccRCC with available follow-up data. Patients who developed recurrence (median follow up=10 months) and those without recurrence (median follow up=65 months) during follow up were identified in an approximately 1:1 ratio. FFPE specimens were retrieved, and a representative region with highest histologic grade from each primary tumor was selected for molecular analyses.

Validation cohort: A query of The Cancer Genome Atlas (TCGA) for patients diagnosed with ccRCC, no evidence of metastasis at diagnosis (M0), and with normalized gene expression and clinical outcomes data available was performed to constitute the validation cohort (n=386). The clinicopathologic characteristics of this cohort have previously been described.⁴

### Validation in the TCGA Cohort

Raw gene expression data were downloaded from TCGA using the R package cgdsr v 1.3.0 and classified using median-centered normalized expression.¹¹ Kaplan-Meier (KM) survival analysis and Cox Proportional-Hazards in R package survival v 3.1-11 were used to interrogate the differences in outcomes of high and low groups.¹² R package fgsea v1.12.0 was used to investigate Gene Set Enrichment Analysis (GSEA) using MSigDB Cancer.¹³ Using expression data from all 386 patients, CCP scores were derived (mxCCP) for each patient, as previously reported and described below.

Epithelial-Mesenchymal Transition (EMT) score derivation: Using the GSEA, 195 genes in the hallmark EMT pathway were identified. Using the validation cohort (TCGA), DEG analysis between ccRCC tumors with and without recurrence was performed to identify differentially expressed EMT genes (LFC)>1 and FDR<5%). Using the same approach as for the mxCCP derivation (see supplement), over-expressed EMT genes were normalized to four housekeeping genes (ATP5E, ARF1, CLTC1, and PGK1) and EMT scores were generated by taking the mean of each EMT retained gene's median-centered expression value to the power of 2, then log 2 transforming the mean.⁸

R package cutpointr was used to derive Youden-Index (J-index) for stratifying CCP and EMT scores into low and high-risk groups for KM survival analysis.²⁸ In TCGA, time to recurrence was used as continuous variable along with recurrence status as categorical variables. In order to derive CCP and EMT Youden-Index cut points, the values at which sensitivity and specificity values are maximized were chosen (Youden Index=sensitivity+specificity−1).

### Molecular Profiling in the Discovery Cohorts

Targeted Next Generation Sequencing (NGS) in the discovery cohorts: RNA was extracted from selected regions of primary tumors (both discovery cohorts) and IVC tumor thrombi (discovery cohort-A only) using the AllPrep FFPE DNA/RNA Kit (Qiagen, Valencia, CA). RNA was quantified using a Qubit 2.0 fluorometer (Thermo Fisher Scientific, Waltham, MA). Multiplex polymerase chain reaction (PCR)-based NGS was performed using a custom kidney cancer targeted panel as previously described^{1,2}. RNAseq gene targets are provided in Table 1.

TABLE 1
Targeted gene panel used to quantify mRNA
expression in discovery cohorts A and B.

| Gene | Type | Target |
|---|---|---|
| ALK | Subtyping | AMPL27472922 |
| MLANA | Subtyping | AMPL28721640 |
| KRT6B | Subtyping | AMPL29984209 |
| KRT6A | Subtyping | AMPL27941400 |
| KRT20 | Subtyping | AMPL30688193 |
| INHA | Subtyping | AMPL8568229 |
| CHGA | Subtyping | AMPL8512809 |
| KRT5 | Subtyping | AMPL6582208 |
| SYP | Subtyping | AMPL11413133 |
| TP63 | Subtyping | AMPL8551205 |
| NCAM1 | Subtyping | AMPL15803418 |
| ESR1 | Subtyping | AMPL1167157 |
| WT1 | Subtyping | AMPL36043510 |
| B3GAT1 | Subtyping | AMPL32272330 |
| CA9 | Subtyping | AMPL6835997 |
| PMEL | Subtyping | AMPL12794306 |
| KIT | Subtyping | AMPL5414183 |
| PGR | Subtyping | AMPL5432601 |
| GATA3 | Subtyping | AMPL5861722 |
| KRT7 | Subtyping | AMPL26896345 |
| MME | Subtyping | AMPL12179698 |
| CTSK | Subtyping | AMPL2641119 |
| CLDN7 | Subtyping | AMPL11137028 |
| KRT19 | Subtyping | AMPL9223556 |
| S100A1 | Subtyping | AMPL29723617 |
| MUC1 | Subtyping | AMPL10434861 |
| POU5F1 | Subtyping | AMPL6780983 |
| TFEB | Subtyping | AMPL10699772 |
| CD163 | Subtyping | AMPL30473871 |
| AMACR | Subtyping | AMPL10004925 |
| CDH16 | Subtyping | AMPL28299802 |
| PAX2 | Subtyping | AMPL28688904 |
| SDHB | Subtyping | AMPL8117849 |
| FH | Subtyping | AMPL1610445 |
| PAX8 | Subtyping | AMPL7740361 |
| TFE3 | Subtyping | AMPL12609698 |
| CCND1 | Subtyping | AMPL16186640 |
| MITF | Subtyping | AMPL20770024 |
| SMARCB1 | Subtyping | AMPL7593839 |
| SDHA | Subtyping | AMPL29183394 |
| KRT18 | Subtyping | AMPL4221330 |
| MDM2 | Subtyping | AMPL7303511 |
| KIF11 | Proliferation | AMPL29149380 |
| RAD54L | Proliferation | AMPL3066603 |
| CENPM | Proliferation | AMPL34960952 |
| CDCA8 | Proliferation | AMPL34142667 |
| SKA1 | Proliferation | AMPL1331432 |
| RAD51 | Proliferation | AMPL10470661 |
| CDC20 | Proliferation | AMPL11753453 |
| PLK1 | Proliferation | AMPL27296096 |
| KIF20A | Proliferation | AMPL28996693 |
| MCM10 | Proliferation | AMPL21941152 |
| DTL | Proliferation | AMPL33518527 |
| BUB1B | Proliferation | AMPL10744784 |
| PBK | Proliferation | AMPL31636523 |
| ORC6 | Proliferation | AMPL24165200 |
| CDK1 | Proliferation | AMPL7721438 |
| TPX2 | Proliferation | AMPL12119161 |
| CEP55 | Proliferation | AMPL4712367 |
| DLGAP5 | Proliferation | AMPL24472126 |
| BIRC5 | Proliferation | AMPL513992 |
| CDCA3 | Proliferation | AMPL15260884 |
| ASF1B | Proliferation | AMPL32091480 |
| MKI67 | Proliferation | AMPL9401897 |
| ASPM | Proliferation | AMPL33832320 |
| FOXM1 | Proliferation | AMPL37179654 |
| CDKN3 | Proliferation | AMPL28832730 |
| PRC1 | Proliferation | AMPL30292251 |
| TOP2A | Proliferation | AMPL3666801 |
| TK1 | Proliferation | AMPL8790684 |
| CENPF | Proliferation | AMPL30585834 |
| RRM2 | Proliferation | AMPL5612106 |
| PTTG1 | Proliferation | AMPL29772431 |
| UBE2C | Proliferation | AMPL12585044 |
| AURKA | Proliferation | AMPL11289894 |
| KIAA0101 | Proliferation | AMPL26246591 |
| MCM4 | Proliferation | AMPL28593509 |
| EZH2 | Proliferation | AMPL27926041 |
| NUSAP1 | Proliferation | AMPL32619751 |
| CDK4 | Proliferation | AMPL4090056 |
| IL6 | Oncotype | AMPL3050298 |
| CEACAM1 | Oncotype | AMPL9839878 |
| NOS3 | Oncotype | AMPL6459737 |
| LMNB1 | Oncotype | AMPL28377520 |
| CX3CL1 | Oncotype | AMPL11539440 |
| TUBB2A | Oncotype | AMPL2876291 |
| CCL5 | Oncotype | AMPL8285121 |
| EDNRB | Oncotype | AMPL9446123 |
| PPAP2B | Oncotype | AMPL8733908 |
| EIF4EBP1 | Oncotype | AMPL27717688 |
| FOXP3 | ImmunoOnc | AMPL25169849 |
| TIGIT | ImmunoOnc | AMPL18574717 |
| LAG3 | ImmunoOnc | AMPL9764508 |
| TNFRSF4 | ImmunoOnc | AMPL9777405 |
| CTLA4 | ImmunoOnc | AMPL28832228 |
| PDCD1 | ImmunoOnc | AMPL29695097 |
| TNFRSF9 | ImmunoOnc | AMPL8469001 |
| CD8B | ImmunoOnc | AMPL16544488 |
| IDO1 | ImmunoOnc | AMPL8392682 |
| PDCD1LG2 | ImmunoOnc | AMPL15271188 |
| PRF1 | ImmunoOnc | AMPL29459941 |
| CD274 | ImmunoOnc | AMPL25431650 |
| CXCL9 | ImmunoOnc | AMPL10447031 |
| CD8A | ImmunoOnc | AMPL17040642 |
| GZMA | ImmunoOnc | AMPL27325297 |
| CXCL10 | ImmunoOnc | AMPL7406108 |
| HAVCR2 | ImmunoOnc | AMPL16031904 |
| TMEM173 | ImmunoOnc | AMPL21621980 |
| HMBS | Housekeeping | AMPL2997752 |
| GPKOW | Housekeeping | AMPL31842971 |
| MRPS9 | Housekeeping | AMPL21275726 |
| AAR2 | Housekeeping | AMPL30761630 |
| ZC3HC1 | Housekeeping | AMPL32597053 |
| AAMP | Housekeeping | AMPL3466806 |
| GPS1 | Housekeeping | AMPL28561615 |
| PSMC1 | Housekeeping | AMPL11245720 |
| DEDD | Housekeeping | AMPL1329250 |
| SLC25A3 | Housekeeping | AMPL27807911 |
| TXNL1 | Housekeeping | AMPL28748937 |
| MMADHC | Housekeeping | AMPL32531518 |
| RPL37 | Housekeeping | AMPL6492146 |
| PSMA1 | Housekeeping | AMPL7464527 |
| PPP2CA | Housekeeping | AMPL9219992 |
| RPL13A | Housekeeping | AMPL12049687 |
| ATP5E | Housekeeping | AMPL12406980 |
| MRFAP1 | Housekeeping | AMPL16011507 |
| ARF1 | Housekeeping | AMPL2170137 |
| CLTC | Housekeeping | AMPL29953281 |
| RPL8 | Housekeeping | AMPL3143652 |
| GPX1 | Housekeeping | AMPL3785890 |
| PGK1 | Housekeeping | AMPL5718916 |
| UBA52 | Housekeeping | AMPL7922207 |
| RPL4 | Housekeeping | AMPL1313288 |
| RPL38 | Housekeeping | AMPL1524005 |
| RPS29 | Housekeeping | AMPL296310 |
| RPLP1 | Housekeeping | AMPL6432001 |
| GAPDH | Housekeeping | AMPL9964196 |

RNA sequencing (RNAseq) analysis: RNAseq reads alignment was performed with Torrent Suite software (Thermo Fisher Scientific, Waltham, MA). Using the coverageAnalysis plugin, end-to-end read counts were generated. Data processing, sample filtering, read normalization, quality control (QC) and downstream analysis were all performed using R (v.3.6.3).³ Samples with <500,000 total mapped reads and/or <60% E2E reads were excluded. For all included samples, log 2-transformed read counts were normalized to the geometric mean of four internally validated housekeeping genes (ATP5E, ARN1, CLTC1, and PGK1).

Differential gene expression and statistical analyses: Differential gene expression data analyses were performed using the R package edgeR v 3.28.1 in discovery and TCGA cohorts.⁴ Log-likelihood ratio testing (LRT) was used to obtain Benjamini-Hochberg False Discovery Rate (FDR) adjusted p-values and fold-changes. The raw data from the two discovery cohorts were imported into R for pre-processing and performed trimmed mean of M-values (TMM) normalization. Batch effects between two cohorts were adjusted for variance using removeBatchEffect function in R package limma for gene expression comparisons. Genes with row sum zero were excluded from all downstream analysis.⁵ 130 amplicons were used to perform differential expression analysis. Differential gene expression was performed in the two discovery cohorts separately. Contrasts were set to perform differential expression: tumor thrombus relative to primary tumor in discovery cohort A (paired analysis); and progressors relative to non-progressors in discovery cohort B. Significance was determined by FDR<5% and −1<log 2 fold-change>1.

Cell Cycle Proliferation score derivation (mxCCP): To analyze cell cycle proliferation, a mxCCP score was derived, based on data used to calculate the commercially available cell cycle proliferation score from Myriad Prolaris™, as previously described.² Briefly, the expression of 31 CCP genes was normalized to the geometric mean of 4 internally well-validated housekeeping genes (ATP5E, ARF1, CLTC1, and PGK1).⁶ The published formula was used for computing CCP scores by taking the mean of each CCP retained gene's median-centered expression value to the power of 2, then log 2 transformed the mean.^{2,7}

### Results

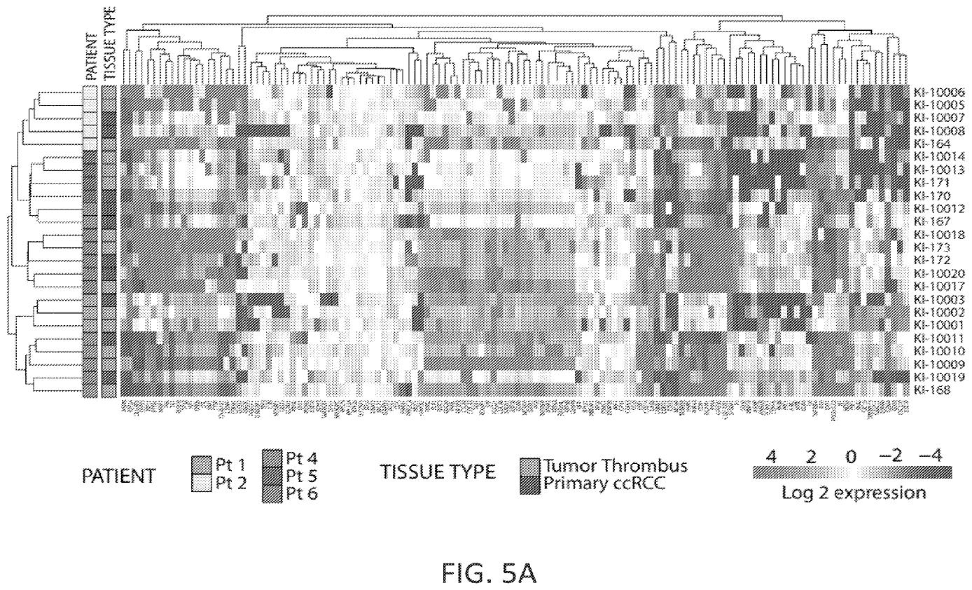

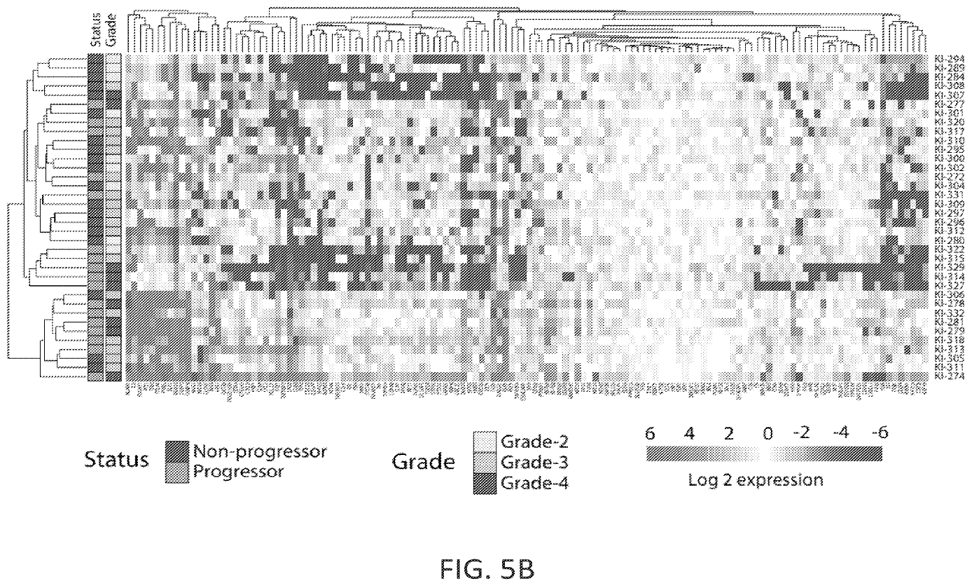

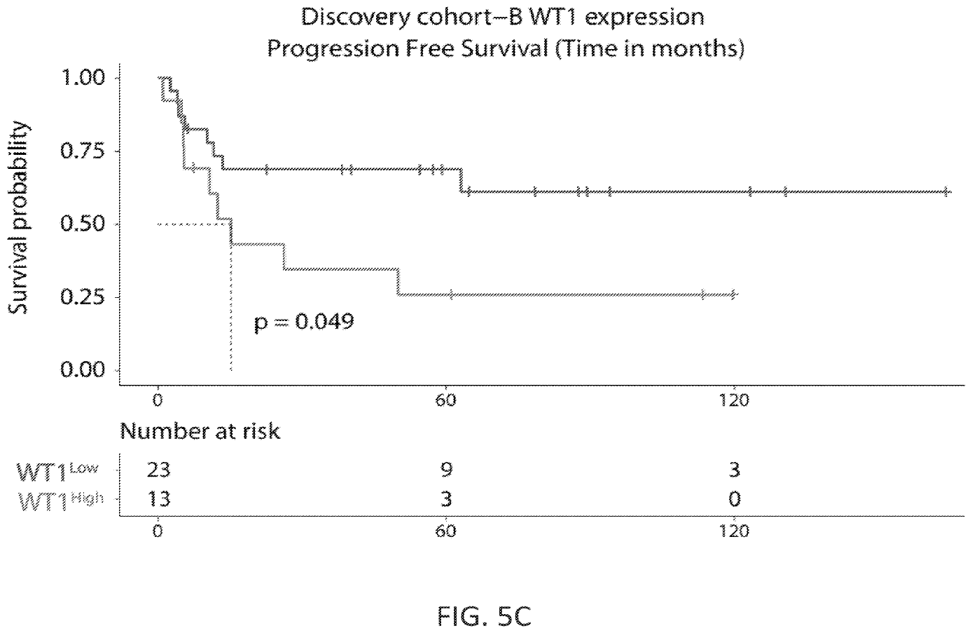

Integrative Molecular Profiling of ccRCC in the Discovery Cohorts Clinicopathologic data on patients in discovery cohort-A are presented in Table 2. In this cohort (FIG. 1a), a total of 30 samples were subjected to targeted RNAseq. Of these, 24 samples passed QC for RNAseq analyses. Unsupervised hierarchical clustering showed heterogeneity between patients, while samples from individual patients typically clustered together (FIG. 5). mxCCP scores for both primary and IVC tumor thrombi were derived.^{8,10} Higher mxCCP scores were observed in tumor thrombi samples vs. primary tumors (mean 6.2 vs. 3.9, p 0.002; FIG. 1b). DEG analysis of RNAseq data across all patients and samples revealed over-expression of nine genes (WT1, FOXM1, GPKOW, AURKA, ORC6, DTL, CENPF, CDKN3, MRPS9) and under-expression of AAR2 in IVC tumor thrombi compared to matched primary tumors (p<0.05; FIG. 1c). WT1 over-expression in the tumor thrombus versus matched primary tumor is shown in FIG. 1d (p=0.007).

TABLE 2
Clinico-pathologic characteristics of patients included in the discovery cohort A (n = 5).

| | Patient 1 | Patient 2 | Patient 4 | Patient 5 | Patient 6 |
|---|---|---|---|---|---|
| Age (years) | 78 | 64 | 76 | 61 | 78 |
| Sex | Female | Male | Male | Female | Female |
| Race | White | White | White | White | White |
| Smoking History | No | Yes | Yes | No | No |
| BMI (kg/m²) | 23 | 23 | 30 | 47 | 24 |
| ECOG PS | 1 | 2 | 0 | 1 | 1 |
| Charlson Comorbidity Index | 3 | 5 | 4 | 3 | 5 |
| Hypertension | Yes | Yes | No | Yes | Yes |
| Presenting symptoms | Yes | Yes | Yes | Yes | Yes |
| TNM stage | T3b N2 | T3c N1 | T3b N0 | T3b N0 | T3c N0 |
| | M0 | M1 | M0 | M0 | M0 |
| T stage | 3b | 3c | 3b | 3b | 3c |
| N stage | 2 | 1 | 0 | 0 | 0 |
| M stage | 0 | 1 | 0 | 0 | 1 |
| Fuhrman Grade | 2 | 3 | 2 | 3 | 3 |
| IVC wall invasion | Yes | Yes | Yes | No | Yes |
| Sarcomatoid histology | No | No | No | No | No |
| Necrosis in histology | Yes | No | Yes | No | No |
| Time to distant recurrence (months) | . | . | 39 | . | 11 |
| Time to death (months) | 17 | 6 | 84 | . | . |

Abbreviations:
BMI = Body mass index,
ECOG PS = Eastern Cooperative Oncology Group Performance Status,
IVC = inferior vena cava

The clinicopathologic data on patients included in discovery cohort-B are presented in Table 3. Of the 36 patients, 17 developed recurrence and 19 had no evidence of recurrence during the follow-up period. (FIG. 1e) mxCCP scores were derived on all patients.^{8,10} contrary to the observation in discovery cohort-A, mxCCP scores were not statistically different between patients with recurrence compared with those without recurrence (mean 2.3 vs. 1.8, p 0.15, Figure if). However, DEG analyses revealed over-expression of UBE2C and CDKN3 and under-expression of CCND1 in patients with recurrence compared to those without recurrence (FDR<5%; FIG. 1g). Consistent with the findings in discovery cohort-A, WT1 over-expression was observed in patients who developed recurrence versus without recurrence (p=0.05, FIG. 1h). KM analysis of PFS revealed that high WT1 expression tumors exhibited shorter PFS compared to low WT1 expression in discovery cohort-B (p=0.049; FIG. 5c).

TABLE 3
Clinico-pathologic characteristics of patients
included in the discovery cohort-B (n = 36).

| | No Recurrence | | Recurrence | | |
|---|---|---|---|---|---|
| | (N = 19) | | (N = 17) | | P-value |
| Age (years) | 55 | [35-79] | 62 | [43-74] | 0.150 |
| Male Gender | 9 | (47) | 11 | (65) | 0.480 |
| Race | | | | | 0.390 |
| African American | 2 | (11) | 0 | (0) | |
| Caucasian | 16 | (84) | 16 | (94) | |
| Unknown | 1 | (5) | 1 | (6) | |
| Fuhrman Grade | | | | | 0.004 |
| 2 | 8 | (42) | 1 | (6) | |
| 3 | 10 | (53) | 8 | (47) | |
| 4 | 1 | (5) | 8 | (47) | |
| T Stage | | | | | 0.002 |
| T1a | 10 | (52) | 0 | (0) | |
| T2 | 3 | (16) | 2 | (12) | |
| T3a | 3 | (16) | 11 | (65) | |
| T3b | 3 | (16) | 4 | (23) | |
| Smoking History | 9 | (47) | 8 | (47) | 0.560 |
| Sarcomatoid Features | 0 | (0) | 4 | (24) | 0.210 |
| Time to Recurrence or | 65 | [6-163] | 10 | [1-63] | 0.420 |
| Last Follow-up (months) | | | | | |

Continuous variables are presented as median [range] and categorical variables are presented as frequency (%).

### Prognostic Significance of CCP and EMT Pathways in the Validation Cohort (TCGA)

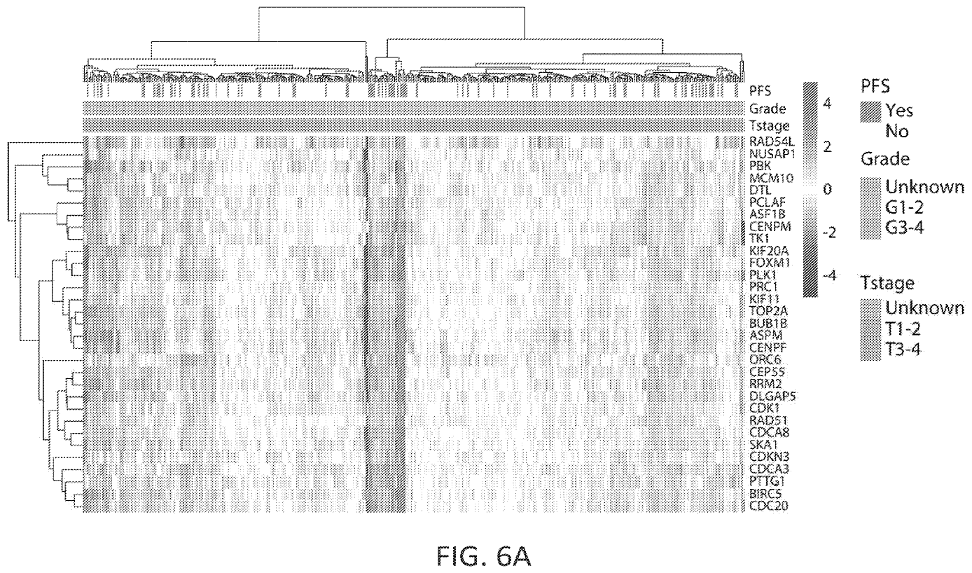

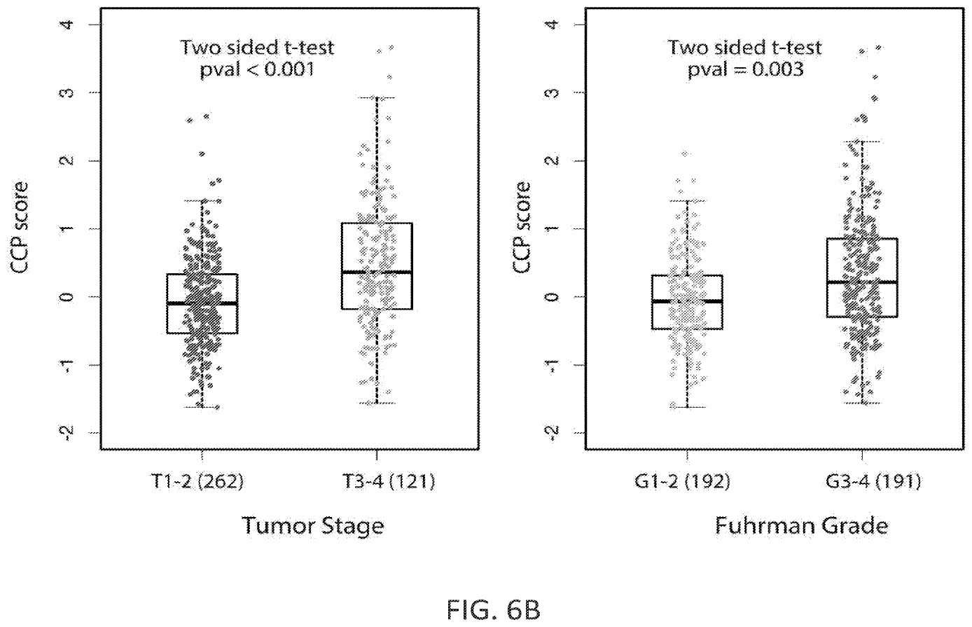

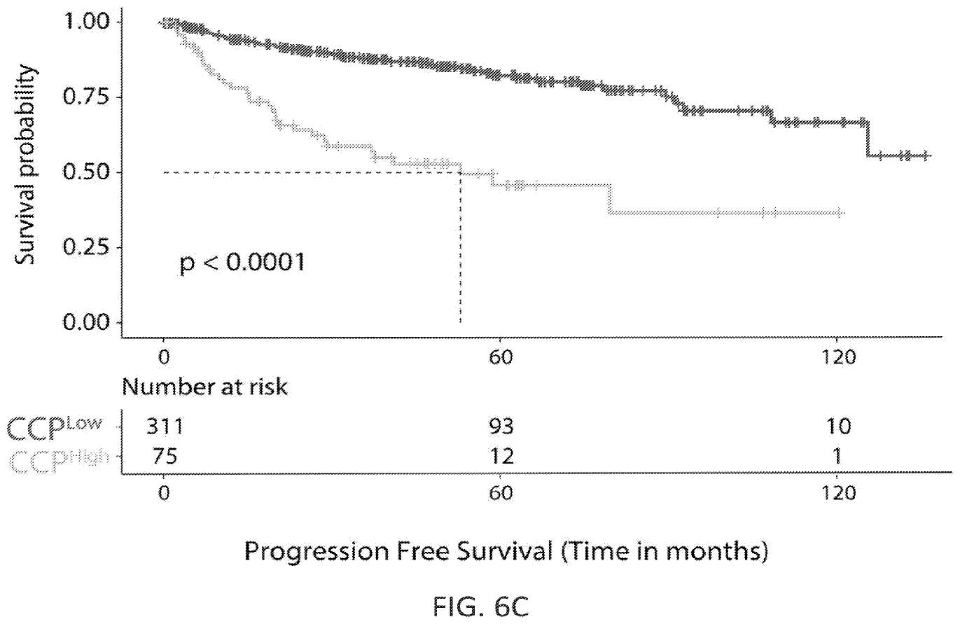

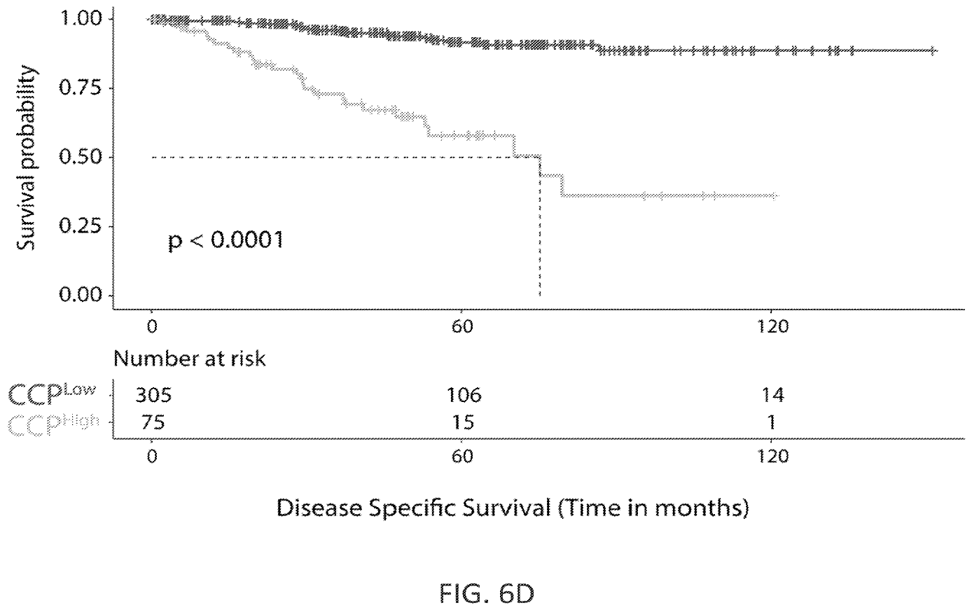

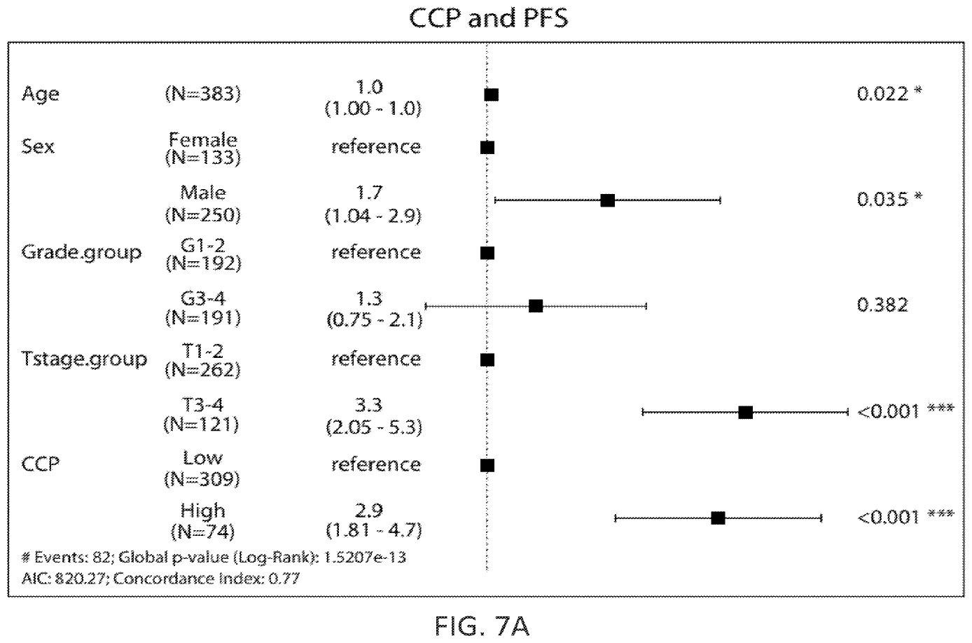

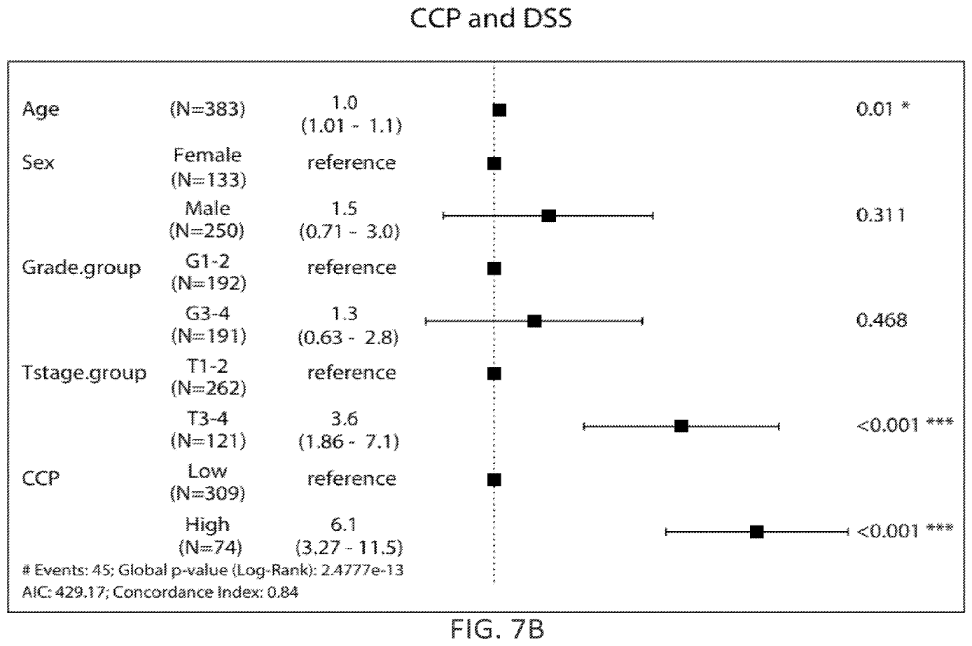

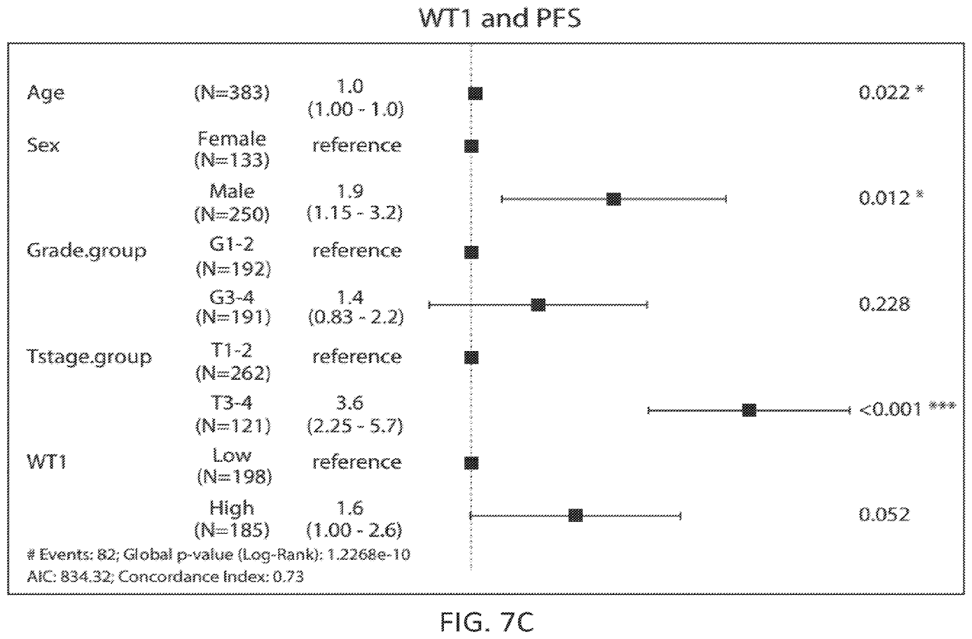

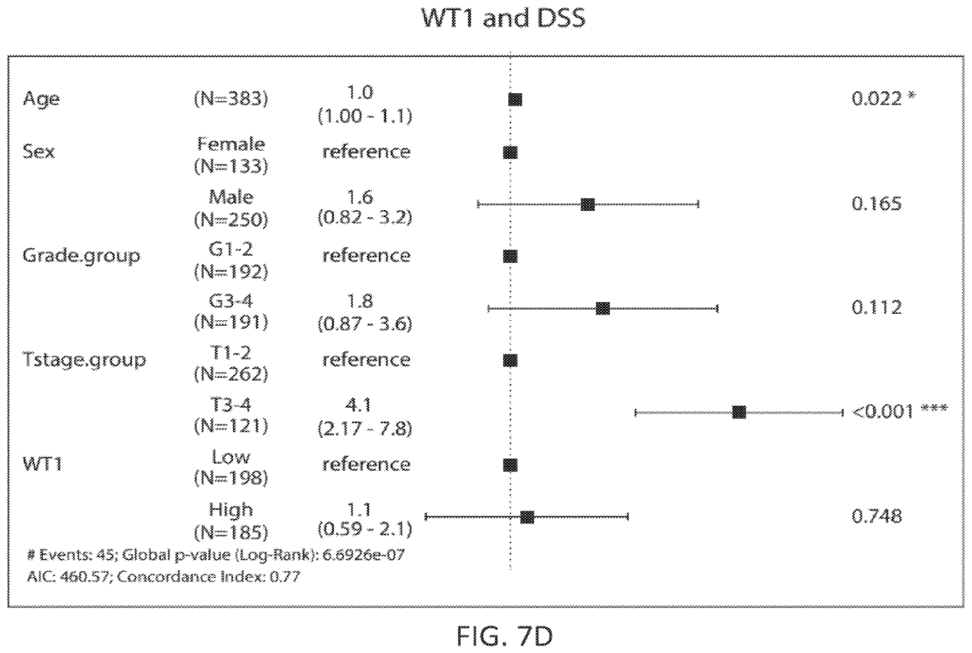

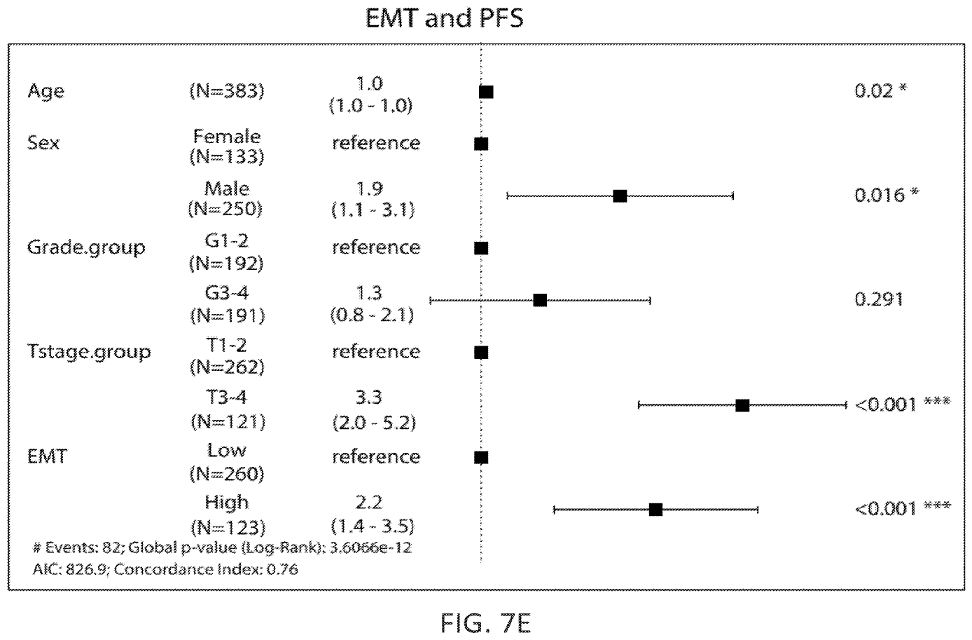

Cell Cycle Proliferation (CCP): Unsupervised hierarchical clustering of the 31 genes used for deriving mxCCP scores revealed a clustering pattern based on disease recurrence and tumor grade (FIG. 6a). The difference in mxCCP scores in localized tumors (pT1/pT2) versus locally advanced tumors (pT3/pT4) was derived and evaluated. A higher mxCCP score amongst pT3/T4 tumors was observed when compared to pT1/T2 tumors (mean 0.37 vs −0.08, p=3.8e-06). Similarly, high grade (Grade 3/4) tumors showed higher mxCCP scores compared with low-grade (Grade 1/2) tumors (mean 0.2 vs −0.05, p=0.002; FIG. 6b). Next, the clinical correlation of mxCCP scores with PFS and DSS was analyzed. The optimal cut-point for mxCCP scores as calculated, which were used to stratify tumors as CCP^{high} (score>0.7; n=75) and CCP^{low} (score≤0.7; n=311). KM analysis of PFS and DSS revealed that patients with CCP^{high} tumors developed earlier disease recurrence and poorer DSS compared to CCP^{low} tumors (p<0.0001; FIG. 6c-d). In a multivariable cox proportional hazard model analyses adjusting for relevant clinicopathologic variables, higher mxCCP scores were independently associated with poorer PFS and DSS (c-index 0.77 and 0.84 respectively, FIG. 7a-b).

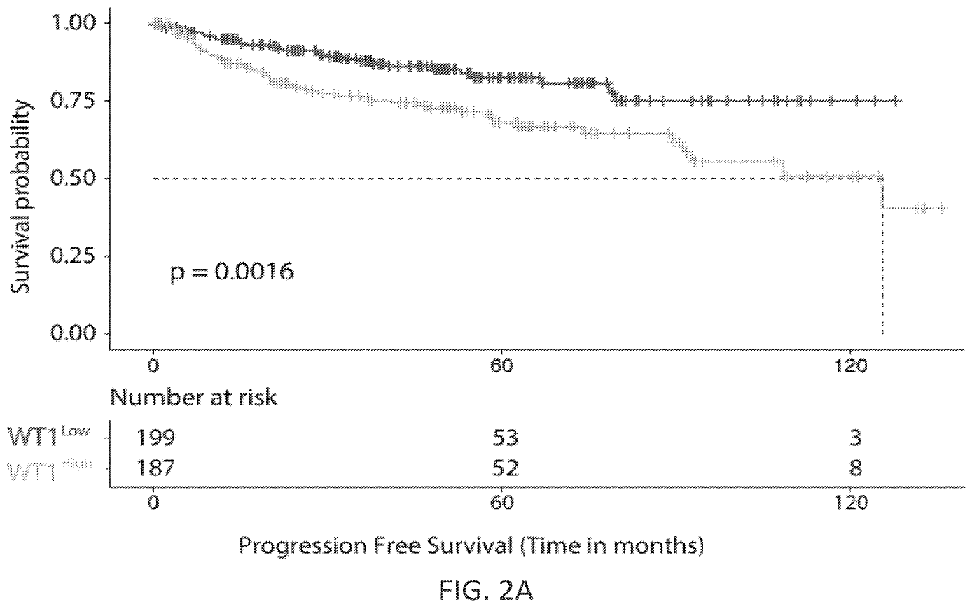

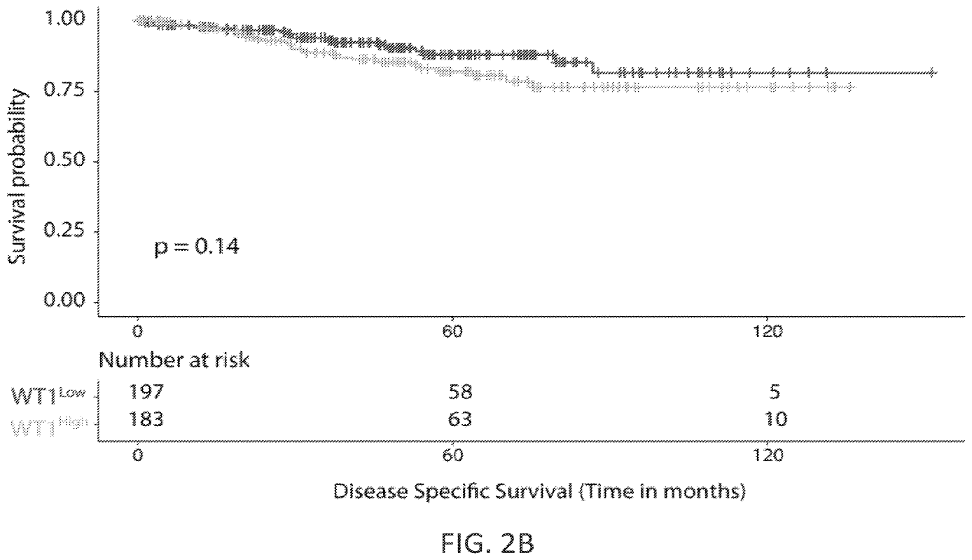

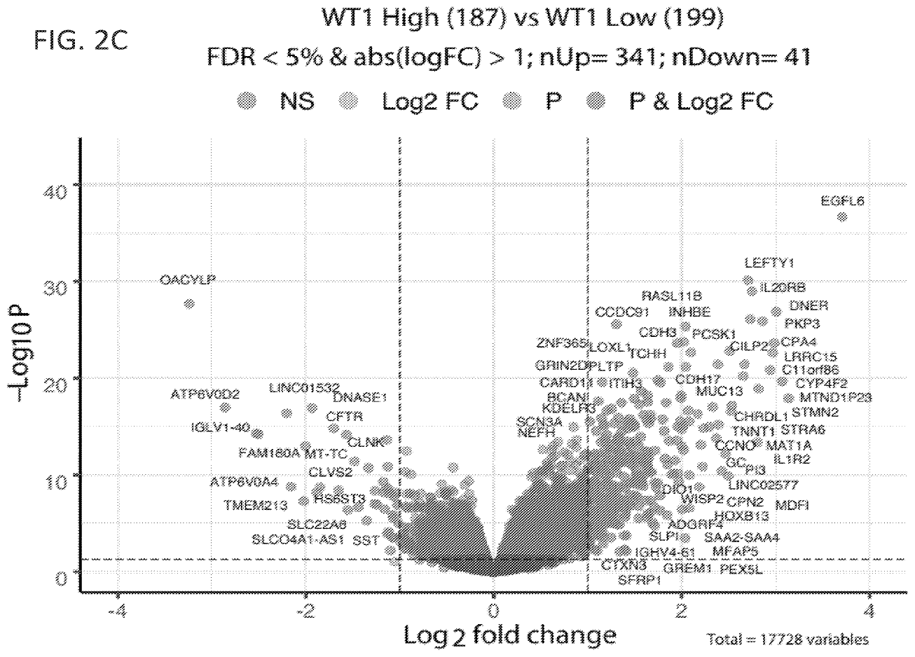

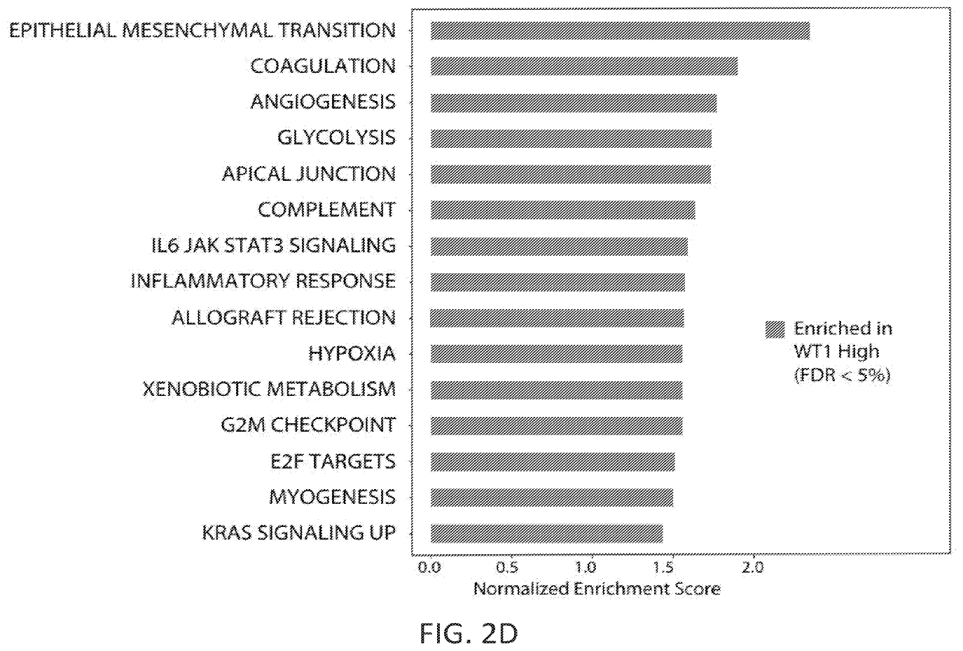

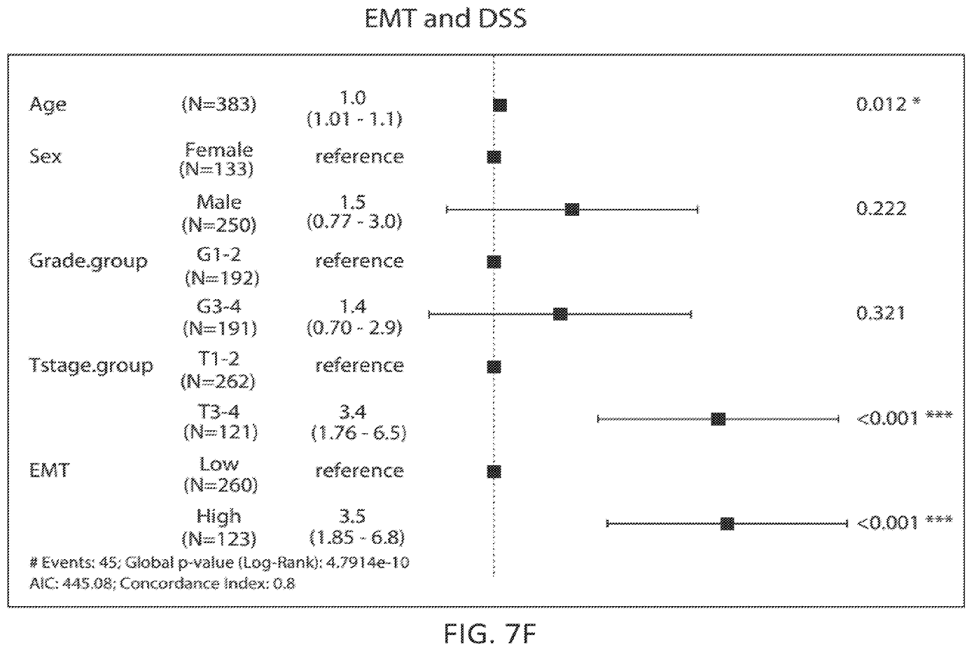

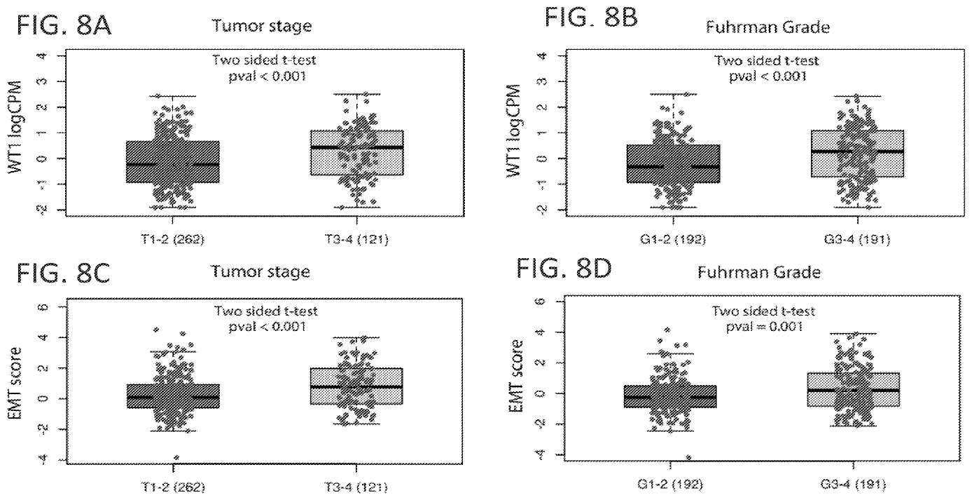

WT1 and Epithelial-Mesenchymal Transition (EMT). To investigate the correlation between advanced or recurrent disease and WT1 expression, raw RNAseq reads on approximately 20,000 genes and clinical outcomes data from our validation cohort was used. The optimal cut-point for WT1 normalized expression values was calculated and tumors were stratified based on WT1 expression as WT1^{high} (expression>0.04; n=187) and WT1^{low} (expression≤0.04; n=199), as described above for CCP score (FIG. 8). To evaluate the clinical significance of high WT1 expression, KM analysis for PFS and DSS was performed. Patients with WT1^{high} tumors exhibited shorter time to disease recurrence but no significant difference in DSS compared to those with WT1^{low} tumors (FIGS. 2a-b). In a multivariable analysis adjusting for relevant clinicopathologic variables, WT1 expression was not significantly associated with PFS or DSS (FIG. 7c-d). After stratifying tumors as WT1^{high} and WT1^{low}, differential expression analysis yielded 382 DEGs (FDR<5% & abs(LFC)>1; FIG. 2c). Hallmark pathway analysis demonstrated EMT pathway as the top statistically significant enriched pathway in WT1^{high} tumors (FDR<5%).

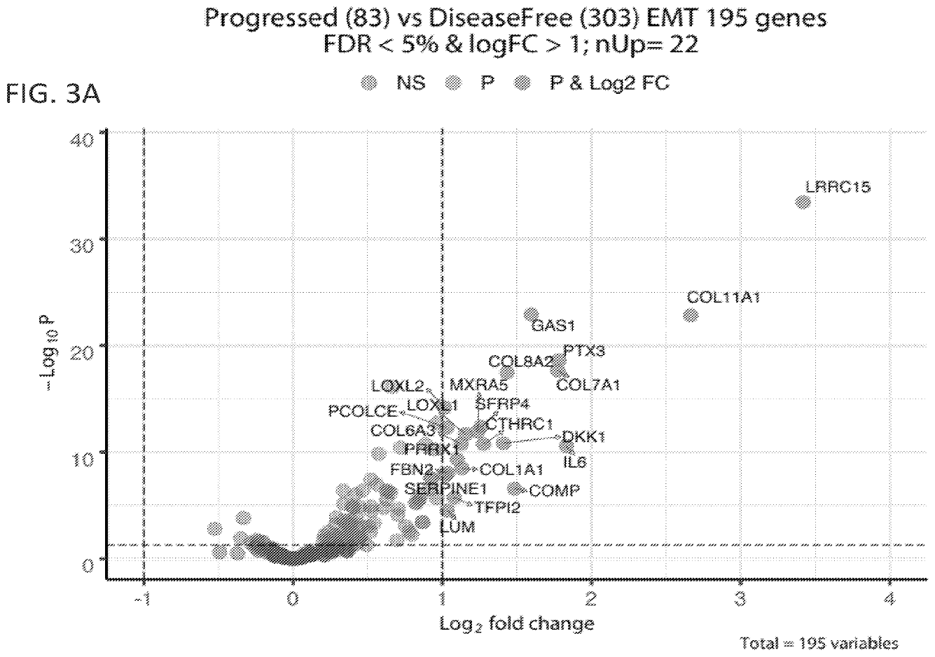

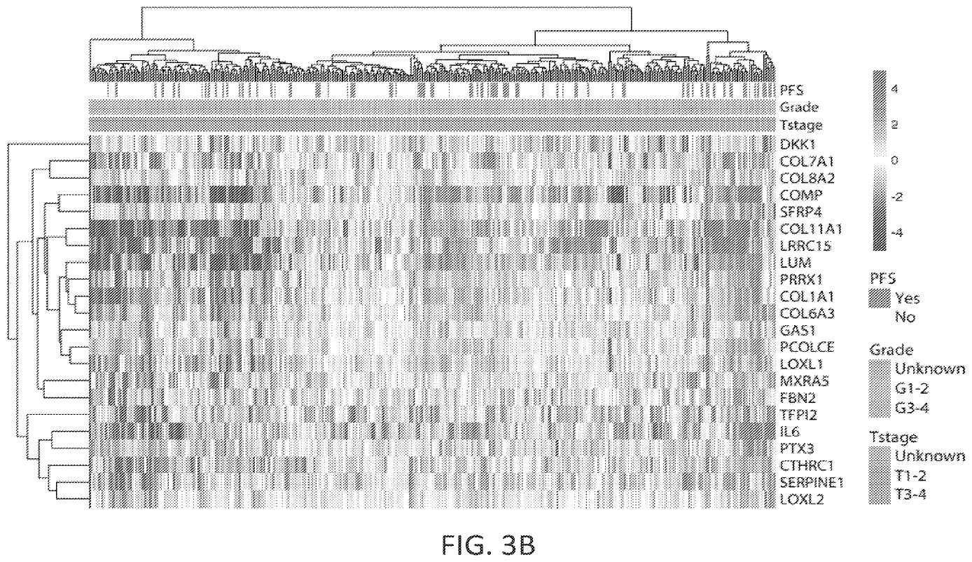

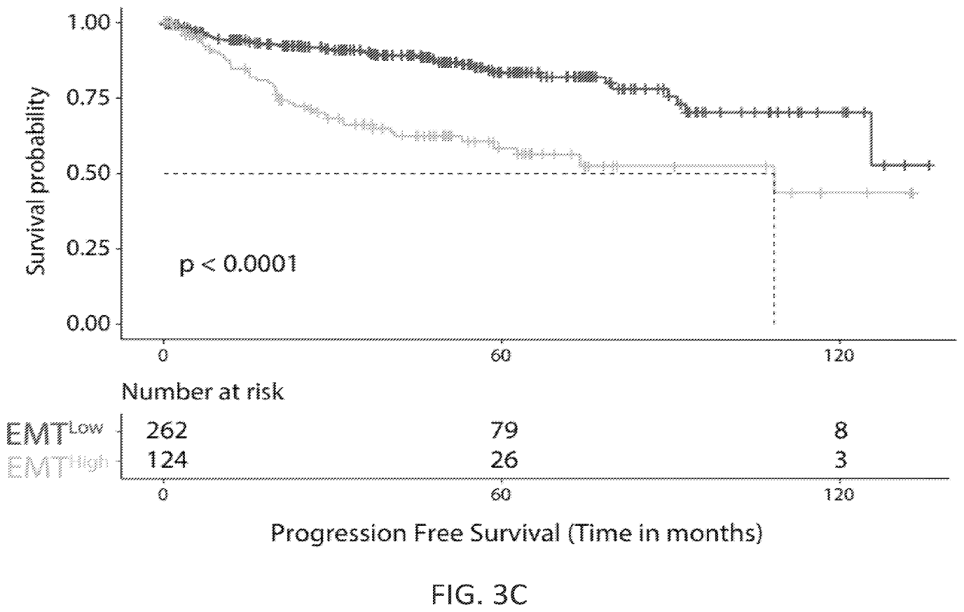

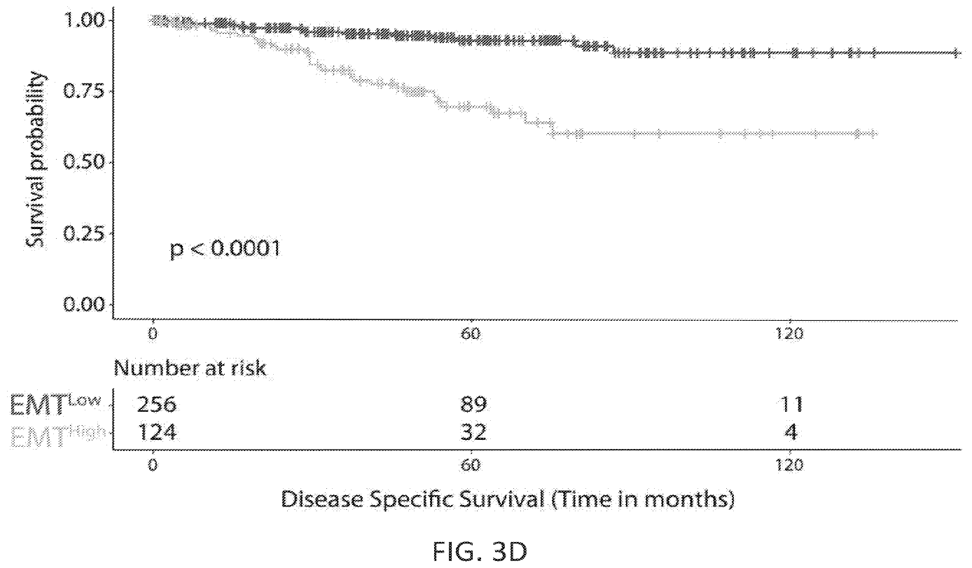

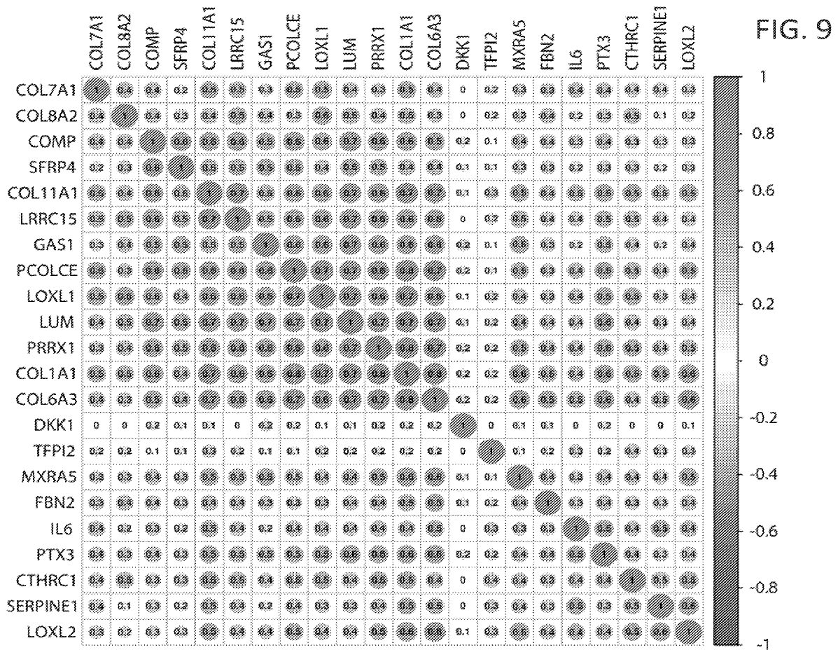

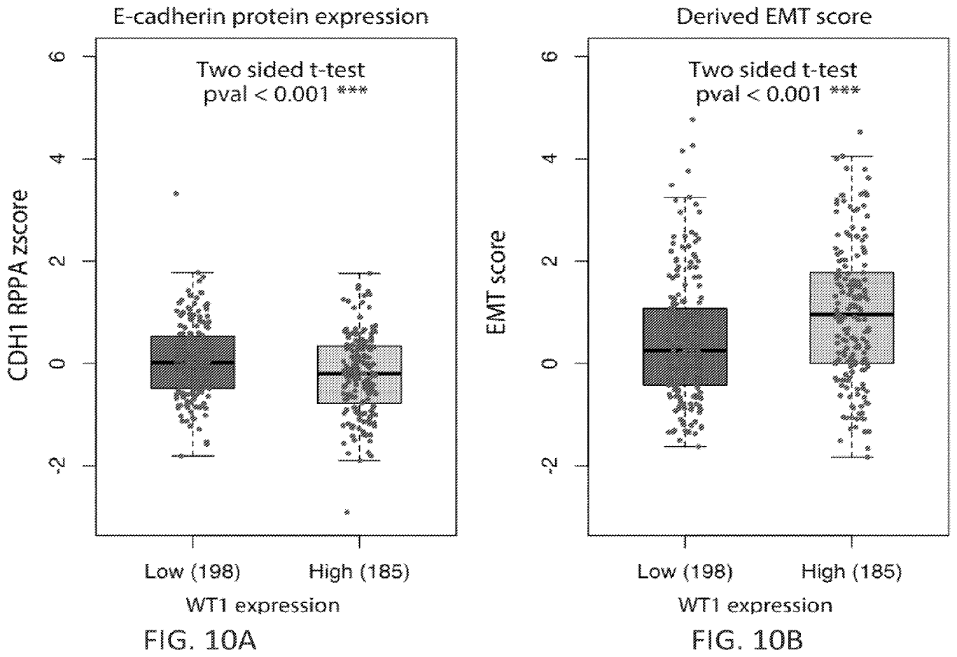

These findings prompted an investigation into the prognostic impact of EMT in ccRCC. Of the 195 identified EMT genes, DEG analysis revealed 22 genes were over-expressed in patients with recurrence in the validation cohort (LFC>1 and FDR<5%; FIG. 3a). Unsupervised hierarchical clustering confirms over-expression of these 22 genes in high grade tumors and in patients with recurrence (FIG. 3b). A positive correlation was observed between each individual gene and the other 22 significant EMT genes (FIG. 9). Consistent with the CCP score derivation approach, a novel EMT score was generated. Tumors were then stratified by optimal cut-point as EMT^{high} (score>1.22; n=124), EMT^{low} (score≤1.22; n=262). Low E-cadherin (CDH1) expression, an indicator of increased EMT enrichment, was observed in patients with high WT1 expression. Additionally, a positive correlation was observed between EMT scores and WT1 expression (p<0.001, FIG. 10). KM analysis revealed poorer PFS and DSS in EMT^{high} compared to EMT^{low} patients (p<0.0001; FIG. 3c-d). In a multivariable analysis adjusting for relevant clinicopathologic variables, EMT^{high} scores were significantly associated with PFS and DSS (c-index 0.76 and 0.8 respectively, FIG. 7e-f).

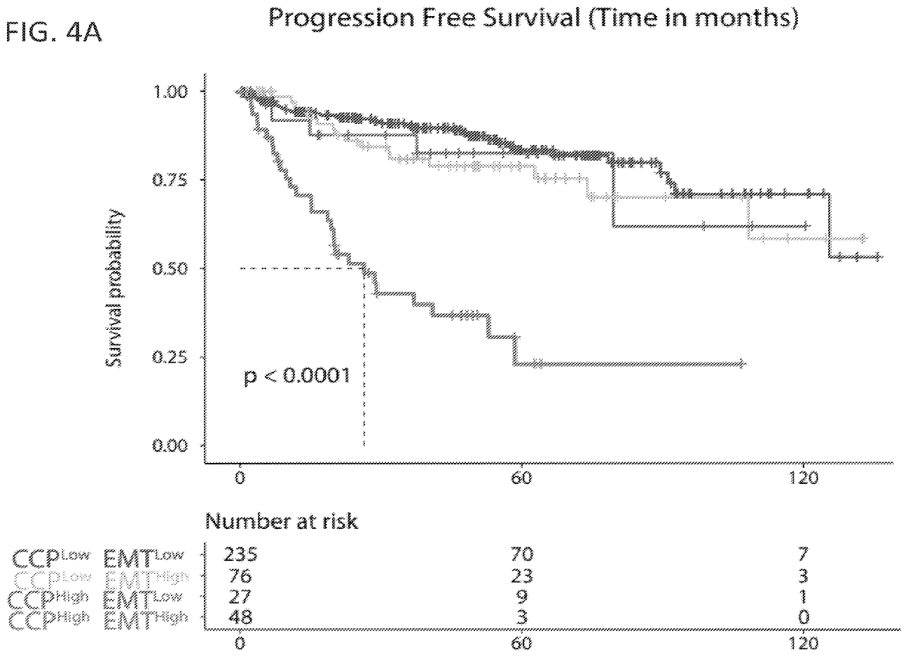

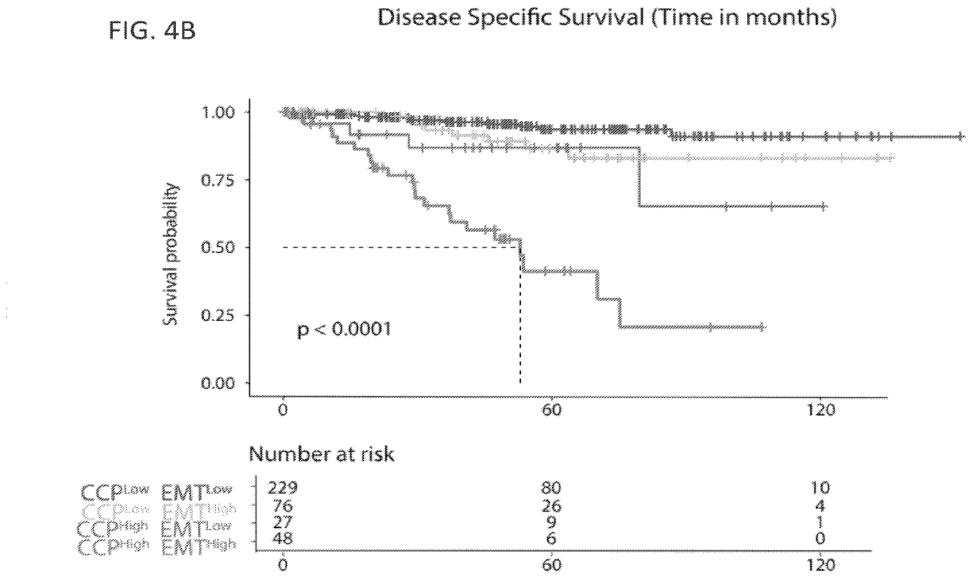

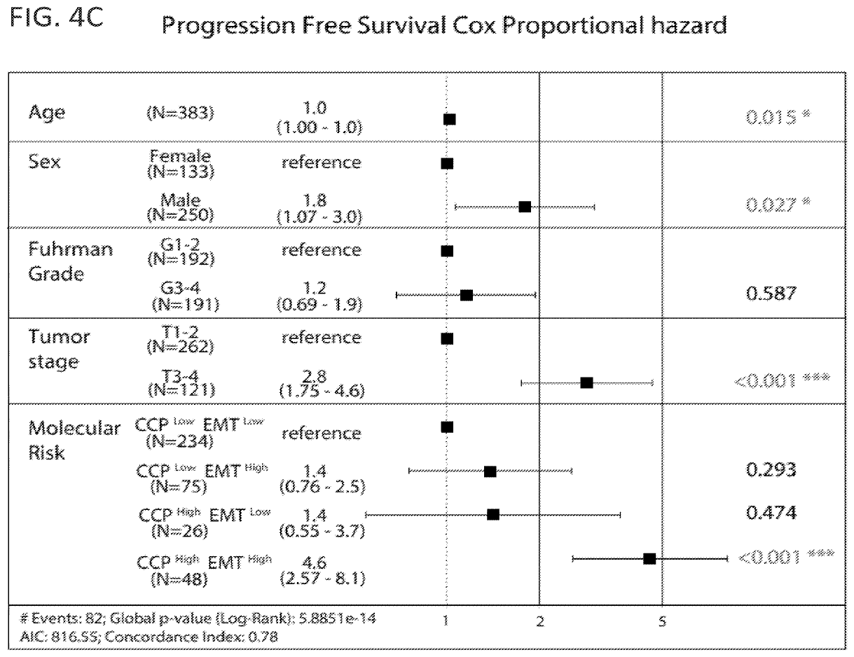

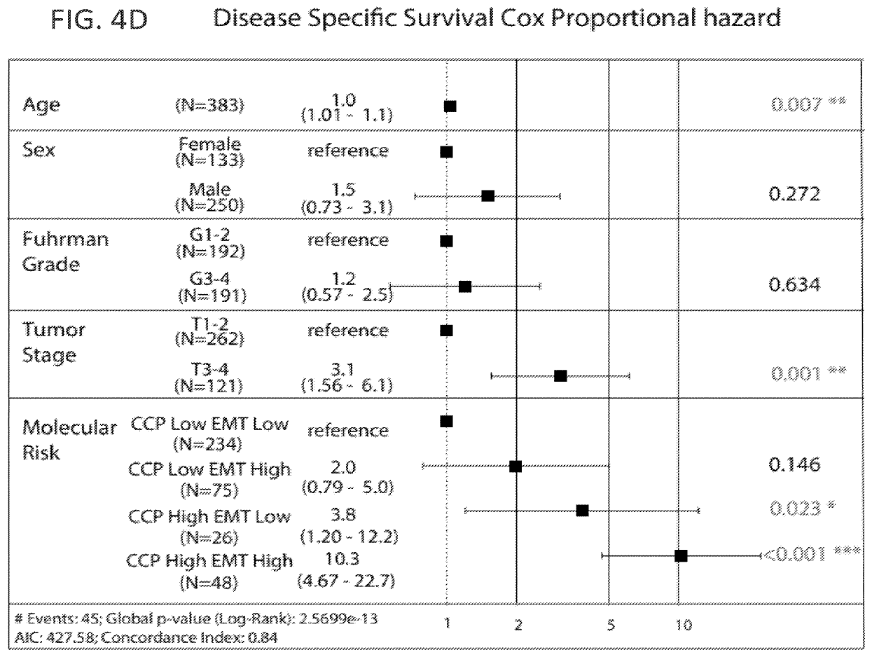

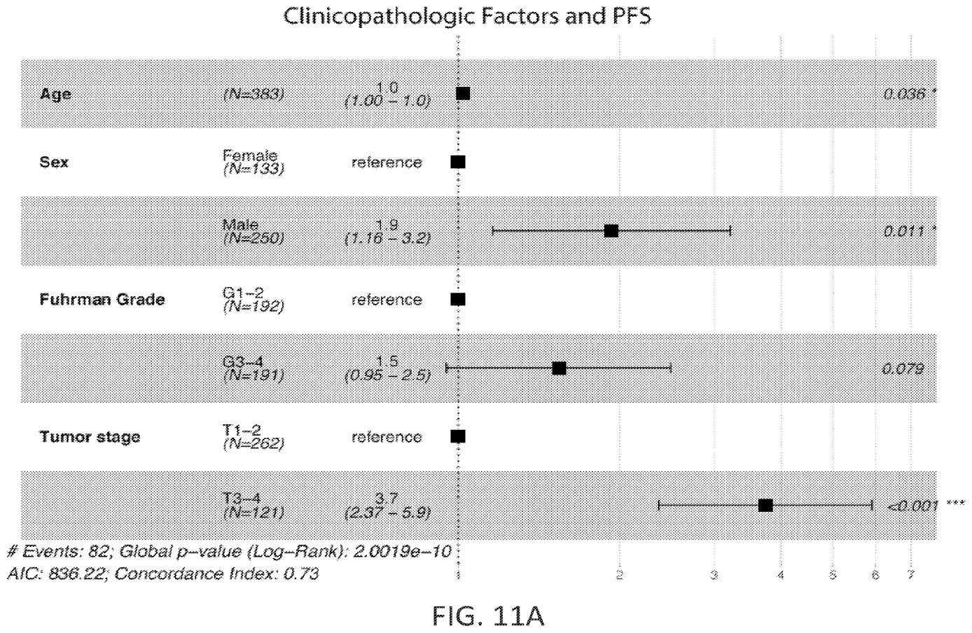

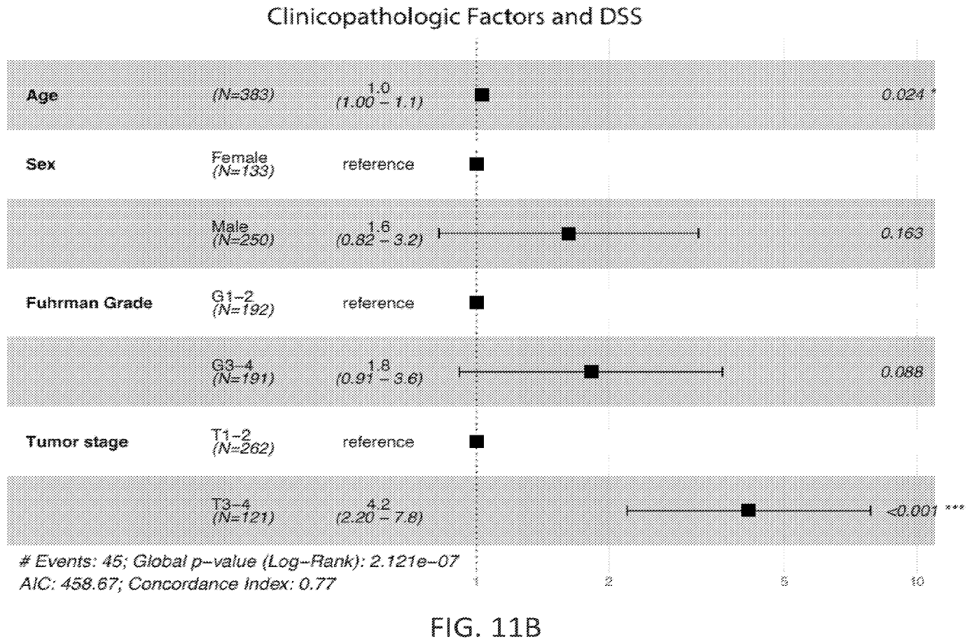

Synergistic Prognostic Implication of CCP and EMT Pathways Enrichment: The above findings demonstrate the prognostic utility of the CCP and EMT pathways, individually. In addition to enrichment of the EMT pathway, this hallmark pathway analysis in WT1^{high} tumors showed enrichment of E2F targets and G2M checkpoints, both CCP pathways (validation cohort, FIG. 2d). The correlation of clinical outcomes with CCP and EMT enrichment, is unknown. Hence, combined derived mxCCP and EMT scores and a Youden cut point index were used to stratify patients into four categories: CCP^{low}/EMT^{low}; CCP^{low}/EMT^{high}; CCP^{high}/EMT^{low}; and CCP^{high}/EMT^{high}. Patients with dual enrichment of CCP and EMT (CCP^{high}/EMT^{high}) exhibited the worst PFS and DSS compared with CCP^{low}/EMT^{low} tumors on KM analysis (FIG. 4a-b). Multivariable analyses adjusting for clinicopathologic variables revealed that CCP and EMT pathways enrichment are independent synergistic predictors of worse PFS and DSS (HR 4.6, 95% CI (2.57-8.1); and 10.3 95% CI (4.67-22.7), respectively; FIG. 4c-d). Notably, addition of CCP and EMT scores to clinicopathologic variables in multivariable Cox proportional hazard models improved the concordance indices from 0.73 to 0.78 for predicting PFS and 0.77 to 0.84 for predicting DSS (FIGS. 4c-d and FIGS. 11A-11B).

### Discussion

Molecular profiling may provide critical prognostic and predictive information for the management of patients with ccRCC. The clinical significance of large-scale gene expression data in ccRCC is poorly understood, resulting in limited clinical implementation. Here, a spectrum of ccRCC ranging from localized disease to locally advanced disease with synchronous IVC tumor thrombi was evaluated to discern molecular pathways implicated in disease progression. Overexpression of WT1, a gene not commonly expressed in adult kidney tissue and possibly involved in EMT, and various CCP genes in IVC tumor thrombi was found compared with matched primary tumors as well as in localized ccRCC with recurrence. An EMT score was developed, the role of EMT and CCP scores in disease recurrence/progression in the TCGA cohort was evaluated. The data herein reveals the independent association and additive impact of EMT and CCP enrichment on PFS and DSS with improvement over clinicopathologic factors alone.

Several studies have examined the role of gene expression across different molecular pathways to risk stratify patients with ccRCC. For example, Consensus Cluster analysis classified ccRCC tumors into two distinct subtypes, clear cell type A (ccA) and B (ccB). Tumors classified as ccA were reported to have significantly improved PFS, cancer-specific survival (CSS), and DSS (all p<0.01) compared to ccB. This classification method, however, was limited by tumor heterogeneity.¹⁴ In another analysis, a 16-gene signature, comprising 11 cancer-specific and 5 reference genes, was developed, validated in separate cohorts and shown to predict RFS, while adjusting for clinicopathologic parameters.^{15,16} Notably, gene sets in these signatures included genes involved in, but not limited to, proliferation, invasion and angiogenesis. These signatures, however, have not been implemented in the routine clinical management of patients with ccRCC.

Cell cycle proliferation (CCP) score has been shown to improve the risk stratification of several cancer types, including breast, prostate, and kidney cancers.^{10,17-19} In an initial report evaluating the CCP score in ccRCC to predict progression to metastatic disease, Askeland et al reported a small improvement in the AUC of clinicopathological variables from 0.78 to 0.84 with the addition of CCP score.⁵ Morgan et al. found that CCP score was an independent predictor of recurrence and disease specific mortality (DSM) after RNx.⁶ However, they reported only a modest improvement in c-index for the CCP-only model compared with the model including CCP plus clinicopathologic variables for predicting DSM (c=0.81 versus 0.85). Additionally, interrogation of the CCP score in the biopsy setting revealed the capacity of this assay to predict adverse pathology after surgery.²⁰ In the current study, derived CCP scores were higher in IVC tumor thrombi compared with primary tumors supporting the association of CCP with aggressive biological behavior. Additionally, derived CCP score was a predictor of PFS and DSS in the validation (TCGA) cohort.

Here, significant over-expression of WT1 and proliferation markers in tumor thrombi compared to paired primary ccRCC as well as in patients with recurrent disease was observed. Moreover, analyses revealed the synergistic role of EMT and CCP pathways enrichment in predicting PFS and DSS. Taken together, these results indicate that the capacity to stratify patients beyond high and low risk, based on a multi-pathway analysis finds use to provide more granular information to identify patients at highest risk for disease recurrence/progression or death.

The compositions and methods provided herein have several clinical and research implications. First, the EMT score finds use to complement the CCP score in identifying patients with ccRCC who may warrant more intense follow-up following RNx. Second, with the increasing use of surveillance for the management of small renal masses, application of the CCP and EMT scores finds use for identification of patients who may not be ideal candidates for surveillance.²⁰ Third, these findings show the critical role of the EMT pathway in the biology of ccRCC progression and provides a pathway for targeted therapies.²¹

### Example 2

The panel of genes and methods of use thereof described herein was further used in patients treated with radical nephrectomy for localized clear cell renal cell carcinoma.

METHODS: Consecutive patients with ccRCC who underwent radical nephrectomy (RNx) for localized disease were retrospectively identified. Those who developed metastasis were identified. Whole-transcriptome mRNA sequencing of primary tumors was performed followed by gene set enrichment analysis (GSEA) for the most significant cancer hallmark pathways enriched in patients who did or did not develop metastasis. For each patient, the 22-gene epithelial mesenchymal transition (EMT) score was calculated (high vs. low), using cut-offs from TCGA data. The prognostic impact of the EMT score was evaluated by performing multivariable cox-proportional hazard testing and Kaplan-Meier (KM) survival analysis.

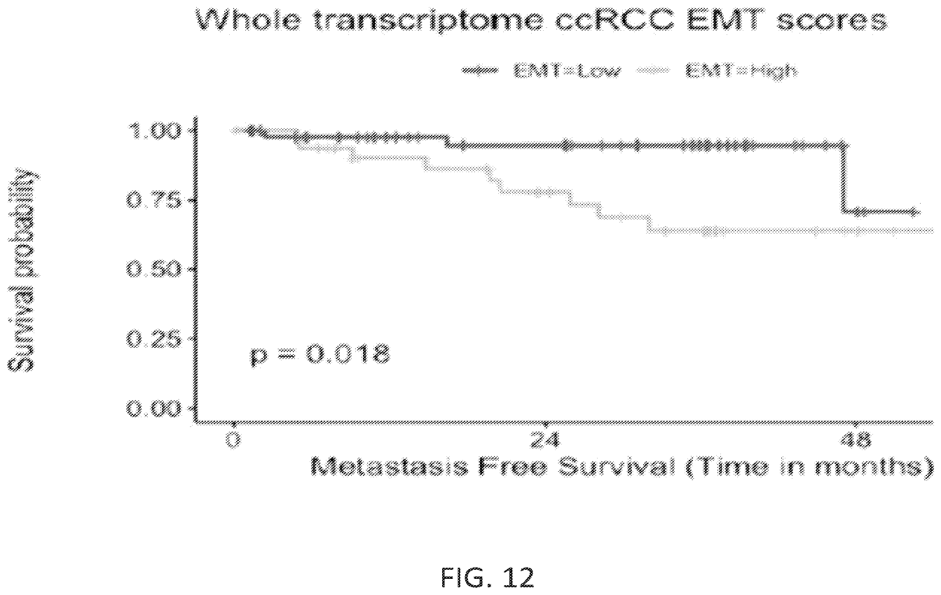

RESULTS: 82 patients with median age 62 years and median tumor size 6+/−2.9 cm were analyzed. The median time to metastasis after radical nephrectomy for patients who developed metastasis (n=12) was 18.2 months and median follow up 31.1 months for patients who did not develop metastasis (n=70). A significant enrichment of EMT, myogenesis, inflammatory response and hypoxia hallmark pathways was observed in patients with metastasis vs. those without metastasis. As shown in FIG. 12, Kaplan-Meier survival analysis showed a significant decrease in metastasis-free survival in patients with a high EMT score (p=0.018). Multivariable analysis controlling for relevant clinicopathologic features such as age, sex, tumor size, tumor stage etc. revealed high EMT score to be significantly associated with development of metastasis [hazard ratio (HR) 7.2; 95% CI 1.15-44.8; p=0.035].

CONCLUSIONS: The 22-gene epithelial mesenchymal transition (EMT) score described herein was validated in patients treated with radical nephrectomy for localized ccRCC. This EMT score improves risk stratification and assists in selecting patients for adjuvant therapy.

### REFERENCES

1. Institute N C. Cancer Stat Facts: Kidney and Renal Pelvis Cancer. https://seer.cancer.gov/statfacts/html/kidrp.html. Published 2019. Accessed.
2. Quencer K B, Friedman T, Sheth R, Oklu R. Tumor thrombus: incidence, imaging, prognosis and treatment. Cardiovasc Diagn Ther. 2017; 7(Suppl 3):S165-S177.
3. Wagner B, Patard J J, Mejean A, et al. Prognostic value of renal vein and inferior vena cava involvement in renal cell carcinoma. Eur Urol. 2009; 55(2):452-459.
4. Cancer Genome Atlas Research N. Comprehensive molecular characterization of clear cell renal cell carcinoma. Nature. 2013; 499(7456):43-49.
5. Askeland E J, Chehval V A, Askeland R W, et al. Cell cycle progression score predicts metastatic progression of clear cell renal cell carcinoma after resection. Cancer Biomark. 2015; 15(6):861-867.
6. Morgan T M, Mehra R, Tiemeny P, et al. A Multigene Signature Based on Cell Cycle Proliferation Improves Prediction of Mortality Within 5 Yr of Radical Nephrectomy for Renal Cell Carcinoma. Eur Urol. 2018; 73(5):763-769.
7. Hovelson D H, McDaniel A S, Cani A K, et al. Development and validation of a scalable next-generation sequencing system for assessing relevant somatic variants in solid tumors. Neoplasia. 2015; 17(4):385-399.
8. Salami S S, Hovelson D H, Kaplan J B, et al. Transcriptomic heterogeneity in multifocal prostate cancer. JCI Insight. 2018; 3(21).
9. Team R C. R: A language and environment for statistical computing. http://www.R-proiect.org/. Published 2013. Accessed.
10. Cuzick J, Swanson G P, Fisher G, et al. Prognostic value of an RNA expression signature derived from cell cycle proliferation genes in patients with prostate cancer: a retrospective study. Lancet Oncol. 2011; 12(3):245-255.
11. Anders Jacobsen A L. R-Based API for Accessing the MSKCC Cancer Genomics Data Server. Computational Biology Center at Memorial-Sloan-Kettering Cancer Center. https://cran.r-project.org/web/packages/cgdsr/cgdsr.pdf Published 2019. Accessed2019.
12. Therneau T M. A Package for Survival Analysis in R. New York: Springer; 2020.
13. Korotkevich G, Sukhov V, Sergushichev A. Fast gene set enrichment analysis. bioRxiv. 2019:060012.
14. Serie D J, Joseph R W, Cheville J C, et al. Clear Cell Type A and B Molecular Subtypes in Metastatic Clear Cell Renal Cell Carcinoma: Tumor Heterogeneity and Aggressiveness. Eur Urol. 2017; 71(6):979-985.
15. Rini B, Goddard A, Knezevic D, et al. A 16-gene assay to predict recurrence after surgery in localised renal cell carcinoma: development and validation studies. Lancet Oncol. 2015; 16(6):676-685.
16. Rini B I, Escudier B, Martini J F, et al. Validation of the 16-Gene Recurrence Score in Patients with Locoregional, High-Risk Renal Cell Carcinoma from a Phase III Trial of Adjuvant Sunitinib. Clin Cancer Res. 2018; 24(18):4407-4415.
17. Lazzeroni M, DeCensi A, Guerrieri-Gonzaga A, et al. Prognostic and predictive value of cell cycle progression (CCP) score in ductal carcinoma in situ of the breast. Mod Pathol. 2020; 33(6):1065-1077.
18. Shangguan X, Qian H, Jiang Z, et al. Cell cycle progression score improves risk stratification in prostate cancer patients with adverse pathology after radical prostatectomy. J Cancer Res Clin Oncol. 2020; 146(3):687-694.
19. Filipits M, Dubsky P, Rudas M, et al. Prediction of Distant Recurrence Using EndoPredict Among Women with ER(+), HER2(−) Node-Positive and Node-Negative Breast Cancer Treated with Endocrine Therapy Only. Clin Cancer Res. 2019; 25(13):3865-3872.
20. Tosoian J J, Feldman A S, Abbott M R, et al. Biopsy Cell Cycle Proliferation Score Predicts Adverse Surgical Pathology in Localized Renal Cell Carcinoma. Eur Urol. 2020.
21. Brett A, Pandey S, Fraizer G. The Wilms' tumor gene (WT1) regulates E-cadherin expression and migration of prostate cancer cells. Mol Cancer. 2013; 12:3.
22. Fraizer G C, Eisermann K, Pandey S, et al. Functional Role of WT1 in Prostate Cancer. In: van den Heuvel-Eibrink M M, ed. Wilms Tumor. Brisbane (AU)2016.
23. Yang L, Han Y, Suarez Saiz F, Minden M D. A tumor suppressor and oncogene: the WT1 story. Leukemia. 2007; 21(5):868-876.
24. Campbell C E, Kuriyan N P, Rackley R R, et al. Constitutive expression of the Wilms tumor suppressor gene (WT1) in renal cell carcinoma. Int J Cancer. 1998; 78(2):182-188.
25. Nakatsuka S, Oji Y, Horiuchi T, et al. Immunohistochemical detection of WT1 protein in a variety of cancer cells. Mod Pathol. 2006; 19(6):804-814.
26. Park J, Kim D H, Shah S R, et al. Switch-like enhancement of epithelial-mesenchymal transition by YAP through feedback regulation of WT1 and Rho-family GTPases. Nat Commun. 2019; 10(1):2797.
27. Piva F, Giulietti M, Santoni M, et al. Epithelial to Mesenchymal Transition in Renal Cell Carcinoma: Implications for Cancer Therapy. Mol Diagn Ther. 2016; 20(2):111-117.
28. Ruopp M D, Perkins N J, Whitcomb B W, Schisterman E F. Youden Index and optimal cut-point estimated from observations affected by a lower limit of detection. Biom J. 2008 June; 50(3):419-30.

We claim:

1. A method comprising determining expression of a panel of genes in a sample obtained from a subject, wherein the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

2. The method of claim 1, wherein the panel of genes comprises at least 6 genes.

3. The method of claim 1, wherein the panel of genes comprises at least 10 genes.

4. The method of claim 1, wherein the panel of genes comprises at least 14 genes.

5. The method of claim 1, wherein the panel of genes comprises at least 18 genes.

6. The method of 1, wherein the panel of genes comprises each of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

7. The method of any one of claims 1-6, wherein the panel of genes comprises less than 50 genes.

8. The method of any one of the preceding claims, further comprising determining expression of one or more genes involved in cell cycle proliferation.

9. The method of claim 8, wherein the one or more genes involved in cell cycle proliferation are selected from FOXM1, ASPM, TK1, PRC1, CDC20, BUB1B, PBK, DTL, CDKN3, RRM2, ASF1B, CEP55, CDC2, DLGAP5, C18orf24, RAD51, KIF11, BIRC5, RAD54L, CENPM, KIAA0101, KIF20A, PTTG1, CDCA8, NUSAP1, PLK1, CDCA3, ORC6L, CENPF, TOP2A, and MCM10.

10. The method of any one of the preceding claims, further comprising determining expression of one or more housekeeping genes in the sample, and normalizing the expression of each member of the panel of genes using the expression of the one or more housekeeping genes.

11. The method of claim 9, wherein the one or more housekeeping genes are selected from ATP5E, ARF1, CLTC1, and PGK1.

12. The method of any one of claims 1-11, wherein the method comprises determining expression of less than 100 genes in total.

13. The method of claim 12, wherein the method comprises determining expression of less than 60 genes in total.

14. The method of any one of the preceding claims, wherein expression of the panel of genes is determined by quantitative PCR (q-PCR).

15. The method of any one of the preceding claims, further comprising assigning an epithelial-mesenchymal transition (EMT) score to the subject based upon the expression of the panel of genes.

16. The method of claim 15, wherein a high EMT score is indicative of increased expression of the panel of genes compared to expression of the equivalent panel of genes for a low EMT score.

17. The method of any one of the preceding claims, wherein the sample is a tumor sample.

18. The method of any one of the preceding claims, wherein the subject is a human.

19. The method of any one of the preceding claims, wherein the subject is suspected of having or at risk of having renal cancer.

20. The method of claim 19, wherein the subject has received a first treatment regimen for renal cancer.

21. The method of claim 20, wherein the first treatment regimen for renal cancer comprises a surgical procedure.

22. The method of any one of claims 19-21, wherein the renal cancer is clear cell renal cell carcinoma.

23. A method of predicting disease outcome in a subject, the method comprising determining expression of a panel of genes in a sample obtained from the subject, wherein the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

24. The method of claim 22, wherein the panel of genes comprises at least 6 genes.

25. The method of claim 22, wherein the panel of genes comprises at least 10 genes.

26. The method of claim 22, wherein the panel of genes comprises at least 14 genes.

27. The method of claim 22, wherein the panel of genes comprises at least 18 genes.

28. The method of claim 22, wherein the panel comprises each of DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2.

29. The method of any one of claims 23-28, wherein the panel of genes comprises less than 50 genes.

30. The method of any one of claims 23-29, further comprising determining expression of one or more housekeeping genes in the sample, and normalizing the expression of each member of the panel of genes using the expression of the one or more housekeeping genes.

31. The method of claim 30, wherein the one or more housekeeping genes are selected from ATP5E, ARF1, CLTC1, and PGK1.

32. The method of any one of claims 23-31, wherein the method comprises determining expression of less than 100 genes in total.

33. The method of claim 32, wherein the method comprises determining expression of less than 60 genes in total.

34. The method of any one of claims 23-33, wherein expression of the panel of genes is determined by quantitative PCR (q-PCR).

35. The method of any one of claims 23-34, comprising predicting a poor disease outcome in the subject when expression of the panel of genes is elevated in the sample.

36. The method of claim 35, wherein a poor disease outcome comprises reduced progression free survival (PFS) and/or disease specific survival (DSS) in the subject.

37. The method of any one of claims 23-36, further comprising assigning an epithelial-mesenchymal transition (EMT) score to the subject based upon the expression of the panel of genes.

38. The method of claim 37, wherein a high EMT score is indicative of increased expression of the panel of genes compared to expression of the equivalent panel of genes for a low EMT score.

39. The method of claim 38, wherein a high EMT score is predictive of reduced progression free survival (PFS) and/or disease specific survival (DSS) in the subject.

40. The method of claim any one of claims 37-39, wherein the EMT score is generated by log₂ transforming the mean expression for each gene in the panel.

41. The method of claim 40, wherein an EMT score above a cutoff value of 1.22 is predictive of reduced progression free survival (PFS) and/or disease specific survival (DSS) in the subject compared to PFS and/or DSS in subjects having EMT scores below the cutoff value.

42. The method of any one of claims 23-41, wherein the subject has received a first treatment regimen for renal cancer.

43. The method of claim 42, wherein the first treatment regimen comprises a surgical procedure.

44. The method of claim 42 or claim 43, wherein the renal cancer is clear cell renal cell carcinoma.

45. The method of any one of claims 23-44, further comprising treating the subject with an aggressive cancer treatment regimen when poor disease outcome is predicted.

46. The method of claim 45, wherein the aggressive cancer treatment regimen comprises one or more therapies selected from radiation therapy, immunotherapy, chemotherapy, targeted therapy, and combinations thereof.

47. A method of treating a subject, the method comprising:
a. determining expression of a panel of genes in a sample obtained from the subject, wherein the panel of genes comprises at least 4 genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2;
b. assigning an epithelial-mesenchymal transition (EMT) score to the subject based upon the expression of the panel of genes, wherein a high EMT score is indicative of increased expression of the panel of genes compared to expression of the equivalent panel of genes for a low EMT score; and
c. treating the subject with an aggressive cancer treatment regimen when the EMT score is above a cutoff value.

48. The method of claim 47, wherein the subject has received a first treatment regimen for renal cancer, wherein the first treatment regimen comprises a surgical procedure.

49. The method of claim 47 or claim 48, wherein the aggressive cancer treatment regimen comprises one or more therapies selected from radiation therapy, immunotherapy, chemotherapy, targeted therapy, and combinations thereof.

50. A kit comprising reagents for detecting one or more genes selected from DKK1, COL7A1, COL8A2, COMP, SRFP4, COL11A1, LRRC15, LUM, PRRX1, COL1A1, COL6A3, GAS1, PCOLCE, LOXL1, MXRA5, FBN2, TFPI2, IL6, PTX3, CTHRC1, SERPINE1, and LOXL2, wherein the kit detects less than 100 genes in total.

51. The kit of claim 45, wherein the kit detects less than 60 genes in total.
